(19) United States
(12) Patent Application Publication
(10) Pub. No.: US 2025/0041166 A1
(43) Pub. Date: Feb. 6, 2025

(54) COSMETIC MATERIAL INK AND COSMETIC SHEET

(71) Applicant: PANASONIC INTELLECTUAL PROPERTY MANAGEMENT CO., LTD., Osaka (JP)

(72) Inventors: Mari UEDA, Osaka (JP); Masato MINAMI, Hyogo (JP); Yuto ENOMOTO, Kanagawa (JP)

(73) Assignee: PANASONIC INTELLECTUAL PROPERTY MANAGEMENT CO., LTD., Osaka (JP)

(21) Appl. No.: 18/924,652

(22) Filed: Oct. 23, 2024

Related U.S. Application Data
(63) Continuation of application No. PCT/JP2023/012258, filed on Mar. 27, 2023.

(30) Foreign Application Priority Data
Apr. 25, 2022 (JP) 2022-071815
Apr. 25, 2022 (JP) 2022-071816

(51) Int. Cl. A61K 8/02

(52) U.S. Cl. CPC A61K 8/0204; A61K 2800/432; A61K 2800/49

(57) ABSTRACT

A cosmetic material ink that is a blue cosmetic material ink containing a blue pigment (A1), acrylic particles (B), an alcohol-based solvent (C), and water (D), in which a content of the blue pigment (A1) is 0.5% by mass or more and 4.5% by mass or less with respect to a total amount of the blue pigment (A1), acrylic particles (B), alcohol-based solvent (C), and water (D), the alcohol-based solvent (C) includes a dihydric alcohol-based solvent (C1) and a trihydric alcohol-based solvent (C2), and a total content of the dihydric alcohol-based solvent (C1) and trihydric alcohol-based solvent (C2) is 30% by mass or more and 85% by mass or less with respect to the total amount of the blue pigment (A1), acrylic particles (B), alcohol-based solvent (C), and water (D).

### FIELD OF INVENTION

The present invention relates to a cosmetic material ink (specifically, a blue cosmetic material ink and a white cosmetic material ink) and a cosmetic sheet obtained by applying the cosmetic material ink by printing.

### BACKGROUND ART

Conventionally, it has been proposed to apply ink containing various kinds of coloring materials to a sheet and affix the coated sheet to the human body, thereby making blemishes, birthmarks, scars (hereinafter also referred to as “discolored areas”) and the like on the skin less noticeable (see, for example, Patent Literature 1). In the technology of Patent Literature 1, an image of the skin is captured and a discolored area is identified. Then, printing is performed on a sheet in a color close to the outer area of the discolored area and then this sheet is affixed to the skin to make the discolored area less noticeable.

However, it is difficult to reproduce the skin color with such a cosmetic sheet. In particular, as a more amount of ink is applied in order to conceal the discolored area, the deviation from the original skin color when the cosmetic sheet is affixed is greater. For this reason, with regard to the ink used in such cosmetic sheets, cosmetic material ink capable of reproducing the original skin color when a cosmetic sheet is affixed has been developed. For example, Patent Literature 2 describes a cosmetic material ink that exhibits high concealing properties for blemishes and the like and is capable of reproducing a color close to the skin color, regardless of the amount of cosmetic material ink applied.

Furthermore, with regard to such a cosmetic material ink, ink for inkjet printing that is highly safe for living bodies, can be stably ejected from an inkjet printer, and affords an image with high durability has also been developed (see, for example, Patent Literature 3).

Patent Literature 1: JP 2015-43836 A

Patent Literature 2: WO 2019/073743 A

Patent Literature 3: JP 2017-57261 A

### SUMMARY OF INVENTION

Accordingly, an object of the present invention is to provide a cosmetic material ink that can be suitably used as a cosmetic material ink and is excellent in ejection stability in inkjet printing.

The present inventors have conducted extensive studies to solve the above problems and, as a result, achieved the present invention. In other words, the present invention includes the following suitable aspects.

A cosmetic material ink according to a first aspect of the present invention is a blue cosmetic material ink comprising:
a blue pigment (A1);
acrylic particles (B);
an alcohol-based solvent (C); and
water (D),
wherein a content of the blue pigment (A1) is 0.5% by mass or more and 4.5% by mass or less with respect to a total amount of the blue pigment (A1), the acrylic particles (B), the alcohol-based solvent (C), and the water (D),
the alcohol-based solvent (C) includes a dihydric alcohol-based solvent (C1) and a trihydric alcohol-based solvent (C2), and
a total content of the dihydric alcohol-based solvent (C1) and the trihydric alcohol-based solvent (C2) is 30% by mass or more and 85% by mass or less with respect to the total amount of the blue pigment (A1), the acrylic particles (B), the alcohol-based solvent (C), and the water (D).

Alternatively, a cosmetic material ink according to another first aspect of the present invention is a white cosmetic material ink comprising:
a white pigment (A2);
acrylic particles (B);
an alcohol-based solvent (C); and
water (D),
wherein the white pigment (A2) has a median diameter of 300 nm or more and 900 nm or less,
a content of the white pigment (A2) is 4% by mass or more and 35% by mass or less with respect to a total amount of the white pigment (A2), the acrylic particles (B), the alcohol-based solvent (C), and the water (D),
the alcohol-based solvent (C) includes a dihydric alcohol-based solvent (C1) and a trihydric alcohol-based solvent (C2), and
a total content of the dihydric alcohol-based solvent (C1) and the trihydric alcohol-based solvent (C2) is 17% by mass or more and 55% by mass or less with respect to the total amount of the white pigment (A2), the acrylic particles (B), the alcohol-based solvent (C), and the water (D).

A cosmetic sheet according to a second aspect of the present invention comprises:
a sheet having a thickness of 10 nm or more and 3000 nm or less; and
a coating film that is formed on the sheet and has at least an ink layer formed of the cosmetic material ink according to a first aspect or the cosmetic material ink according to another first aspect.

### DETAILED DESCRIPTION

As described above, cosmetic material inks and cosmetic material inks for inkjet printing having various kinds of performance have been developed in recent years. Meanwhile, for example, when discolored areas are concealed and/or skin color is expressed, cosmetic material inks of a plurality of colors are often used by being mixed or applied to a sheet. Hence, regarding each color of the cosmetic material ink, it would be preferable if the various kinds of performance of the cosmetic material ink could be further improved.

As a result of extensive studies, the present inventors have found out that a blue cosmetic material ink can be suitably used to express skin color by setting the content (% by mass) of the blue pigment contained in the blue cosmetic material ink to be in a predetermined range. The present inventors have also found out that by setting the median diameter and content (% by mass) of the white pigment contained in a white cosmetic material ink to be in predetermined ranges, a cosmetic sheet produced using the white cosmetic material ink exhibits favorable concealing properties for discolored areas and is excellent in ejection stability in inkjet printing.

Furthermore, the present inventors have found out that a cosmetic material ink exerts excellent ejection stability in inkjet printing by containing both a dihydric alcohol-based solvent and a trihydric alcohol-based solvent as an alcohol-based solvent contained in a blue or white cosmetic material ink (in the present specification, also referred to as “blue cosmetic material ink or white cosmetic material ink”, or simply “cosmetic material ink”) and setting the total content (% by mass) of the dihydric alcohol-based solvent and trihydric alcohol-based solvent to be in a predetermined range.

As described above, the cosmetic material ink of the present embodiment can be suitably used as a cosmetic material ink and is excellent in ejection stability in inkjet printing.

More specifically, according to the present embodiment, it is possible to provide a blue cosmetic material ink that can be suitably used to express skin color and is excellent in ejection stability in inkjet printing. Alternatively, according to the present embodiment, it is possible to provide a white cosmetic material ink that exhibits favorable concealing properties for discolored areas and is excellent in ejection stability in inkjet printing.

In the present specification, the “coating film” formed on a substrate, for example, a sheet, includes not only a coating film having only an ink layer formed a blue or white cosmetic material ink, but also a coating film having an ink layer formed of a blue or white cosmetic material ink and one or more ink layers formed of cosmetic material inks of other colors in a laminated state.

In the present specification, the “coating film having a spectral reflectance that can be (sensorily) recognized as skin color” means a coating film in which the curve obtained by measuring the spectral reflectance of the coating film formed using a blue or white cosmetic material ink and a cosmetic material ink of another color at wavelengths of 400 nm to 700 nm satisfies all of the following requirements (i) to (iv) (see Patent Literature 2).
(i) The first inflection point exists in a wavelength region of less than 550 nm.
(ii) The second inflection point exists in a wavelength region of 550 nm or more and 610 nm or less.
(iii) The spectral reflectance at the second inflection point is higher than the spectral reflectance at the first inflection point.
(iv) The spectral reflectances in a wavelength region of more than 580 nm are all higher than the spectral reflectance at a wavelength of 580 nm.

In the present specification, “expressing skin color” or “exhibiting skin color expressiveness” means that a coating film formed using a cosmetic material ink has a spectral reflectance that can be sensorily recognized as skin color, or that a coating film is determined to exhibit suitable skin color expressiveness when the skin color expressiveness of a cosmetic material ink is evaluated by the method described in Examples later. Specifically, the latter refers to a case where the color difference ΔE*ab between the measured values when the L* value, a* value, and b* value in the L*a*b* color system of a coating film formed using a blue cosmetic material ink, a red cosmetic material ink, and a yellow cosmetic material ink are measured using a spectrophotometer and the measured values of the L* value, a* value, and b* value of a skin model is less than 13.0.

Alternatively, in the present specification, “expressing skin color” means that a coating film formed using a cosmetic material ink has a spectral reflectance that can be sensorily recognized as skin color, or that a coating film formed using a cosmetic material ink satisfies the conditions for the L* value, a* value, and b* value presented below.

### (Conditions for L* Value, a* Value, and b* Value)

In a case where the L* value, a* value, and b* value in the L*a*b* color system of a coating film formed using a white cosmetic material ink and a cosmetic material inks of another color are measured using a spectrophotometer (for example, “CM-700d” manufactured by Konica Minolta, Inc.), the color difference ΔE*ab between the measured values in this case and the measured values of the L* value, a* value, and b* value of a skin model (for example, BIOSKIN (Product Model Number: BIO manufactured by Beaulax Co., Ltd.)) is less than 13.0.

Hereinafter, embodiments of the present invention will be described in detail. The scope of the present invention is not limited to the embodiments described herein, and various modifications can be made without departing from the spirit of the present invention.

### 1. Cosmetic Material Ink (Blue Cosmetic Material Ink or White Cosmetic Material Ink)

The cosmetic material ink in the present embodiment, that is, the blue cosmetic material ink or the white cosmetic material ink, is excellent in ejection stability in inkjet printing. For this reason, the cosmetic material ink is used, in particular, as an ink for inkjet printing, and is applied to a substrate by printing. Specifically, the blue or white cosmetic material ink is preferably an ink for inkjet printing to be applied to a sheet having a thickness of 10 nm or more and 3000 nm or less by inkjet printing. Such a thin sheet serving as a substrate will be described in detail later.

Furthermore, as will be described in more detail later, a coating film having an ink layer formed of the blue or white cosmetic material ink is formed on such a thin sheet, for example, by inkjet printing to produce a cosmetic sheet. Alternatively, a coating film having an ink layer formed of the blue or white cosmetic material ink and one or more ink layers formed of cosmetic material inks of other colors may be formed on a sheet to produce a cosmetic sheet.

### 1-1. Blue Cosmetic Material Ink

First, among the cosmetic material inks in the present embodiment, the blue cosmetic material ink will be described in detail below.

### 1-1-1. Respective Ingredients of Blue Cosmetic Material Ink

The blue cosmetic material ink in the present embodiment contains a blue pigment (A1), acrylic particles (B), an alcohol-based solvent (C), and water (D). Furthermore, the blue cosmetic material ink may contain ingredients other than these if necessary. Hereinafter, the respective ingredients will be described in detail.

### <Blue Pigment (A1)>

The blue pigment (A1) contained in the blue cosmetic material ink is not particularly limited as long as it is a pigment capable of producing a blue color, but is preferably a pigment selected from ingredients listed in the list of cosmetic ingredient display names based on the Pharmaceutical Affairs Law of Japan, ingredients that comply with the EU Cosmetics Directive (Cosmetics Directive 76/768/EEC), or ingredients listed in the International Cosmetic Ingredient Dictionary and Handbook (Jan. 1, 2002, 9th edition) by the US CTFA (Cosmetic, Toiletry & Fragrance Association, U.S.) from the viewpoint of skin irritation. Specifically, the blue pigment (A1) may be either of an inorganic pigment or an organic pigment used in arbitrary cosmetic products and the like known to those skilled in the art, but is preferably an inorganic pigment from the viewpoint of being suitably used without adversely affecting the skin.

Specifically, as such an inorganic pigment, the blue pigment (A1) preferably includes at least one selected from Prussian blue (ferric ferrocyanide), ultramarine blue, lapis lazuli, blue verditer, aluminum-cobalt oxide, aluminum-zinc-cobalt oxide, silicon-cobalt oxide, silicon-zinc-cobalt oxide, cobalt pigment, smalt, cobalt blue, cobalt stannate, cobalt chrome blue, cobalt-aluminum-silicon oxide, and manganese blue.

Among these, the blue pigment (A1) more preferably includes Prussian blue (ferric ferrocyanide) from the viewpoints of color producing properties, safety, and biocompatibility as a cosmetic material.

The content of the blue pigment (A1) is 0.5% by mass or more and 4.5% by mass or less with respect to the total amount of the blue pigment (A1), acrylic particles (B), alcohol-based solvent (C), and water (D).

When the content of the blue pigment (A1) is less than 0.5% by mass, it is difficult to impart the blue color derived from the pigment to the substrate, and it becomes difficult to express skin color by using the blue cosmetic material ink together with cosmetic material inks of other colors. When the content of the blue pigment (Al) exceeds 4.5% by mass, the blue color derived from the pigment is excessively imparted to the substrate, and it becomes difficult to express skin color by using the blue cosmetic material ink together with cosmetic material inks of other colors.

The content of the blue pigment (A1) is preferably 0.52% by mass or more, more preferably 1% by mass or more, still more preferably 2% by mass or more. The content of the blue pigment (A1) is preferably 4.15% by mass or less, more preferably 4% by mass or less, still more preferably 3% by mass or less.

The blue pigment (A1) preferably has a median diameter (the median value (D50) of integrated values in particle size distribution) measured by a laser diffraction method of 90 nm or more and 500 nm or less. By setting the median diameter of the blue pigment (A1) to 90 nm or more, it becomes easy to impart the blue color derived from the pigment to the substrate, and it becomes easy to express skin color by using the blue cosmetic material ink together with cosmetic material inks of other colors. By setting the median diameter of the blue pigment (A1) to 500 nm or less, the blue color derived from the pigment is prevented from being excessively produced on the substrate, and it becomes easy to express skin color by using the blue cosmetic material ink together with cosmetic material inks of other colors. By setting the median diameter of the blue pigment (A1) to be in such as range, the blue cosmetic material ink can be stably ejected from an inkjet printer.

The median diameter of the blue pigment (A1) is more preferably 95 nm or more, still more preferably 100 nm or more. The median diameter of the blue pigment (A1) is more preferably 400 nm or less, still more preferably 250 nm or less.

### <Acrylic Particles (B)>

The blue cosmetic material ink in the present embodiment contains acrylic particles (B). The acrylic particles (B) fix the blue cosmetic material ink to the substrate and improve the water resistance and durability of the printed matter.

The acrylic particles (B) are not particularly limited as long as they are particles formed of a (meth)acrylic resin having the above-mentioned action, but examples thereof include an alkyl (meth)acrylate polymer, a styrene/(meth)acrylic copolymer, an alkyl (meth)acrylate/vinyl acetate copolymer, a (meth)acrylic acid/alkyl (meth)acrylate copolymer, and an alkyl (meth)acrylate dimethicone copolymer. In the present specification, (meth)acrylic refers to acrylic, methacrylic, or a mixture of acrylic and methacrylic.

The acrylic particles (B) are preferably particles formed of a (meth)acrylic resin that does not irritate to the skin. For this reason, the acrylic particles (B) are preferably selected from ingredients listed in the list of cosmetic ingredient display names based on the Pharmaceutical Affairs Law of Japan, ingredients that comply with the EU Cosmetics Directive (Cosmetics Directive 76/768/EEC), or ingredients listed in the International Cosmetic Ingredient Dictionary and Handbook (Jan. 1, 2002, 9th edition) by the US CTFA (Cosmetic, Toiletry & Fragrance Association, U.S.). Specifically, it is more preferable to select the acrylic particles (B) from particles that are formed of an arbitrary (meth)acrylic resin known to those skilled in the art and already used in cosmetics and the like.

Specific examples of the acrylic particles (B) include a homopolymer of a (meth)acrylic monomer, a copolymer of two or more kinds of (meth)acrylic monomers, and a copolymer of a (meth)acrylic monomer and another monomer.

Among these, the acrylic particles (B) preferably contain at least one of a homopolymer of a (meth)acrylic monomer and a copolymer of two or more kinds of (meth)acrylic monomers. The acrylic particles (B) more preferably contain an acrylates copolymer. An acrylates copolymer is a copolymer composed of two or more kinds of the monomers among acrylic acid, methacrylic acid, and their simple esters.

Examples of the (meth)acrylic monomer include acrylic acid, methyl acrylate, ethyl acrylate, acrylic acid amide, n-propyl acrylate, n-butyl acrylate, isobutyl acrylate, octyl acrylate, 2-ethylhexyl acrylate, N,N-dimethylaminoethyl acrylate, acrylonitrile, methacrylic acid, ethyl methacrylate, methacrylic acid amide, n-propyl methacrylate, n-butyl methacrylate, isobutyl methacrylate, 2-ethylhexyl methacrylate, octyl methacrylate, hydroxyethyl methacrylate, and N,N-dimethylaminoethyl methacrylate.

Examples of another monomer copolymerizable with the (meth)acrylic monomers include styrene, vinyl acetate, a silicone macromer, a fluorine-based monomer, and an alkoxysilane unsaturated monomer.

The content of the acrylic particles (B) is not particularly limited, but is preferably 1% by mass or more and 20% by mass or less with respect to the total amount of the blue pigment (A1), acrylic particles (B), alcohol-based solvent (C), and water (D).

By setting the content of the acrylic particles (B) to be in such as range, it is possible to secure the effect, such as favorable fixity (water resistance), of the blue cosmetic material ink.

The content of the acrylic particles (B) is more preferably 1.5% by mass or more, still more preferably 2% by mass or more. The content of the acrylic particles (B) is more preferably 15% by mass or less, still more preferably 10% by mass or less.

The median diameter of the acrylic particles (B) is preferably 1 nm or more and 150 nm or less. The median diameter of the acrylic particles (B) means the median value (D50) of integrated values in the particle size distribution measured by a laser diffraction method. It is still more preferable that the 90% value (D90) of the integrated values in the particle size distribution of the acrylic particles (B) is 250 nm or less. By setting the particle size of the acrylic particles (B) to be in such as range, the fixity (water resistance) of the blue cosmetic material ink can be further improved and the blue cosmetic material ink can be more stably ejected from an inkjet printer.

When the blue cosmetic material ink is prepared, the acrylic particles (B) are mixed with the blue pigment (A1), alcohol-based solvent (C), water (D) and the like usually in a state of being dispersed in a dispersion medium (slurry or dispersion state). The dispersion medium at this time is preferably a solvent that does not irritate the skin, and is preferably an alcohol-based solvent (C) or water (D) described below.

### <Alcohol-Based Solvent (C)>

The blue cosmetic material ink in the present embodiment contains an alcohol-based solvent (C). The alcohol-based solvent (C) has the functions as a moisturizer (anti-drying agent) and a solvent for the blue cosmetic material ink. In detail, the alcohol-based solvent (C) prevents the area around the nozzles of an inkjet printer from drying out and suppresses clogging due to the adhesion of ink and the like when inkjet printing is performed using the blue cosmetic material ink.

The alcohol-based solvent (C) is not particularly limited as long as it is a higher alcohol that has 3 or more carbon atoms and exhibits the above-mentioned functions, but is preferably one that does not irritate the skin. The alcohol-based solvent (C) is absorbed into the substrate or volatilizes after the blue cosmetic material ink is applied to the substrate by printing.

The alcohol-based solvent (C) preferably has 3 to 5 carbon atoms, more preferably has 3 or 4 carbon atoms. In a case where the alcohol-based solvent (C) has carbon atoms in such a range, the alcohol-based solvent (C) becomes more compatible with water (D) to be described later.

The alcohol-based solvent (C) preferably includes an alcohol-based solvent having a boiling point of 200° C. or more. By containing the alcohol-based solvent (C) having a boiling point of 200° C. or more, the moisturizing action can be further enhanced, and the area around the nozzles of an inkjet printer can be more reliably prevented from drying out. In this case, it is more preferable that both a dihydric alcohol-based solvent (C1) and a trihydric alcohol-based solvent (C2), which will be described later, include an alcohol-based solvent having a boiling point of 200° C. or more.

The blue cosmetic material ink in the present embodiment contains both the dihydric alcohol-based solvent (C1) and trihydric alcohol-based solvent (C2) as the alcohol-based solvent (C).

By containing both the dihydric alcohol-based solvent (C1) and trihydric alcohol-based solvent (C2), the blue cosmetic material ink can be stably ejected from an inkjet printer. In other words, in a case where only either of the dihydric alcohol-based solvent (C1) or the trihydric alcohol-based solvent (C2) is contained as the alcohol-based solvent (C), ejection defects such as ejection deflection may occur when inkjet printing is performed.

By containing the dihydric alcohol-based solvent (C1) as the alcohol-based solvent (C), the viscosity and surface tension of the blue cosmetic material ink can be prevented from becoming excessively high and the ink can be stably ejected from an inkjet printer. The wettability of the blue cosmetic material ink with respect to the substrate can be improved and unevenness is less likely to occur in the finally formed coating film.

The dihydric alcohol-based solvent (C1) preferably contains at least one selected from diethylene glycol, propylene glycol, 1,3-propanediol, butylene glycol, and hexanediol. Among these, 1,3-propanediol is more preferable from the viewpoint of suppressing aggregation of the blue pigment.

By containing the trihydric alcohol-based solvent (C2) as the alcohol-based solvent (C), the alcohol-based solvent (C) and/or water (D) to be described later can be less likely to volatilize and the viscosity of the blue cosmetic material ink can be stably maintained in a range suitable for inkjet printing. As a result, the ink can be stably ejected from an inkjet printer.

The trihydric alcohol-based solvent (C2) is preferably glycerin from the viewpoint of being highly safe for living bodies. In a case where glycerin is contained as the trihydric alcohol-based solvent (C2), the pigment can be less likely to aggregate and the ink can be less likely to thicken excessively even when stored for a long period of time.

The total content of the dihydric alcohol-based solvent (C1) and trihydric alcohol-based solvent (C2) is 30% by mass or more and 85% by mass or less with respect to the total amount of the blue pigment (A1), acrylic particles (B), alcohol-based solvent (C), and water (D).

When the total content of the dihydric alcohol-based solvent (C1) and trihydric alcohol-based solvent (C2) is in such a range, the viscosity of the blue cosmetic material ink can be stably maintained in a range suitable for inkjet printing. When the viscosity of the blue cosmetic material ink is in a range suitable for inkjet printing, the shapes of flying droplets after ink ejection can be stabilized, the droplet shapes when the droplets land on a substrate can be uniformized, and ink ejection deflection can be prevented.

When the total content of the dihydric alcohol-based solvent (C1) and trihydric alcohol-based solvent (C2) is in such a range, the area around the nozzles of an inkjet printer can be favorably moisturized. As a result, nozzle clogging and print defects due to drying are not caused, and the blue cosmetic material ink can be stably ejected from an inkjet printer. Specifically, when the total content of the dihydric alcohol-based solvent (C1) and trihydric alcohol-based solvent (C2) exceeds 85% by mass, the pigment aggregates and this is likely to lead to ink ejection failure due to nozzle clogging.

The total content of the dihydric alcohol-based solvent (C1) and trihydric alcohol-based solvent (C2) is preferably 35% by mass or more, more preferably 37% by mass or more, still more preferably 40% by mass or more. The total content of the dihydric alcohol-based solvent (C1) and trihydric alcohol-based solvent (C2) is preferably 80% by mass or less, more preferably 65% by mass or less, still more preferably 50% by mass or less. In other words, the total content of the dihydric alcohol-based solvent (C1) and trihydric alcohol-based solvent (C2) is particularly preferably 40% by mass or more and 50% by mass or less.

The content of the dihydric alcohol-based solvent (C1) is preferably 15% by mass or more and 45% by mass or less with respect to the total amount of the blue pigment (A1), acrylic particles (B), alcohol-based solvent (C), and water (D). By setting the content of the dihydric alcohol-based solvent (C1) to be in such a range, it is easier to stably maintain the viscosity of the blue cosmetic material ink in a range suitable for inkjet printing and the area around the nozzles of an inkjet printer can be more favorably moisturized. As a result, a blue cosmetic material ink superior in ejection stability in inkjet printing can be obtained.

The content of the dihydric alcohol-based solvent (C1) is more preferably 17% by mass or more, still more preferably 20% by mass or more. The content of the dihydric alcohol-based solvent (C1) is more preferably 42% by mass or less, still more preferably 30% by mass or less. In other words, the content of the dihydric alcohol-based solvent (C1) is particularly preferably 20% by mass or more and 30% by mass or less.

The content of the trihydric alcohol-based solvent (C2) is also preferably 15% by mass or more and 45% by mass or less with respect to the total amount of the blue pigment (A1), acrylic particles (B), alcohol-based solvent (C), and water (D). By setting the content of the trihydric alcohol-based solvent (C2) to be in such a range, it is easier to stably maintain the viscosity of the blue cosmetic material ink in a range suitable for inkjet printing and the area around the nozzles of an inkjet printer can be more favorably moisturized. As a result, a blue cosmetic material ink superior in ejection stability in inkjet printing can be obtained.

The content of the trihydric alcohol-based solvent (C2) is more preferably 17% by mass or more, still more preferably 20% by mass or more. The content of the trihydric alcohol-based solvent (C2) is more preferably 42% by mass or less, still more preferably 30% by mass or less. In other words, the content of the trihydric alcohol-based solvent (C2) is particularly preferably 20% by mass or more and 30% by mass or less.

### <Water (D)>

The blue cosmetic material ink in the present embodiment contains water (D), preferably purified water. Water (D) exerts the function as a solvent for the blue cosmetic material ink. Water (D) is absorbed into the substrate or volatilizes after the blue cosmetic material ink is applied to the substrate by printing.

Water (D) is not particularly limited as long as it is one generally used in cosmetics. For example, water (D) may be purified water that has been purified by various methods such as distillation and ion exchange. Examples of water (D) may include hot spring water, deep sea water, and steam distilled water from plants.

The content of water (D) is preferably 10% by mass or more and 60% by mass or less, more preferably 50% by mass or more and 60% by mass or less, still more preferably 50% by mass or more and 56% by mass or less with respect to the total amount of the blue pigment (A1), acrylic particles (B), alcohol-based solvent (C), and water (D). By setting the content of water (D) to be in such a range, the viscosity of the blue cosmetic material ink can be likely to fall in a range suitable for inkjet printing.

### <Other Ingredients>

The blue cosmetic material ink may further contain various kinds of additives and the like in a range in which the effects, such as skin color expressiveness and the ejection stability in inkjet printing, of the blue cosmetic material ink in the present embodiment are not impaired.

For example, the blue cosmetic material ink may contain a surfactant as an additive.

When a surfactant is contained as an additive, the surfactant aids in stable dispersion of the pigment in the ink, and aggregation and settling of the blue pigment is thus suitably suppressed. The kind of surfactant is not particularly limited, but a nonionic surfactant or an anionic surfactant is preferable from the viewpoint that electrostatic aggregation of the blue pigment can be suppressed. Examples of such a surfactant include phosphoric acid-based surfactants, phosphates, sucrose-based surfactants, and carboxylates.

The additive is preferably a compound that does not irritate the skin. Examples of other additives include pH adjusters, thickeners, ultraviolet absorbers, UV scattering agents, preservative and fungicides, oxygen scavengers, antioxidants, preservatives, anti-fading agents, antifoaming agents, fragrances, and solvents other than the alcohol-based solvent (C) and water (D).

### 1-1-2. Method for Preparing Blue Cosmetic Material Ink

The blue cosmetic material ink in the present embodiment can be prepared by, for example, mixing a slurry or dispersion containing the blue pigment (A1), acrylic particles (B), alcohol-based solvent (C), and water (D) and, if necessary, other ingredients using a disperser, or the like. For mixing of the respective ingredients, an arbitrary method known to those skilled in the art may be applied. For example, mixing can be carried out using a ball mill, a sand mill, a roll mill, a homomixer, an attritor, or the like.

### 1-1-3. Physical Properties of Blue Cosmetic Material Ink

It is preferable that the blue cosmetic material ink in the present embodiment has a viscosity of 3 mPa·s or more and 15 mPa·s or less at 25° C. when the viscosity is measured at a shear rate of 1000 (1/s) using a cone-plate viscometer. As the viscosity of the blue cosmetic material ink is in such a range, the blue cosmetic material ink can be more stably ejected from an inkjet printer. Specifically, the shapes of flying droplets after ink ejection can be stabilized, the droplet shapes when the droplets land on a substrate can be uniformized, and ink ejection deflection can be prevented.

The viscosity of the blue cosmetic material ink when measured under the same conditions as above is more preferably 3.5 mPa·s or more, still more preferably 4 mPa·s or more. The viscosity of the blue cosmetic material ink when measured under the same conditions as above is more preferably 10 mPa·s or less, still more preferably 6 mPa·s or less.

The pH of the blue cosmetic material ink is preferably 7 to 10. As the pH of the blue cosmetic material ink is in such a range, erosion of members such as the nozzles of various printers by the blue cosmetic material ink, and the like can be suppressed. When the pH of the blue cosmetic material ink is in such a range, the pigment is less likely to aggregate in a case where the blue cosmetic material ink is stored for a long period of time, and the blue cosmetic material ink can be thus stably ejected from an inkjet printer.

The surface tension of the blue cosmetic material ink at 25° C. is more preferably 30 mN/m or more and 40 mN/m or less. As the surface tension of the blue cosmetic material ink at 25° C. is in such a range, wettability of the blue cosmetic material ink becomes favorable, and thus a coating film having a uniform thickness can be formed when the blue cosmetic material ink is ejected from an inkjet printer onto a substrate. In the present specification, the surface tension is a value measured by a pendant drop method.

The median diameter of the particles contained in the blue cosmetic material ink (the median value (D50) of integrated values in particle size distribution) measured by a laser diffraction method is preferably 1 nm or more and 200 nm or less. It is preferable that the 90% value (D90) of the integrated values in the particle size distribution of the particles contained in the blue cosmetic material ink is 250 nm or less. By setting the D50 or preferably the D50 and D90 of the particles contained in the blue cosmetic material ink to be in such ranges, the blue cosmetic material ink can be more stably ejected from an inkjet printer.

The D50 of the particles contained in the blue cosmetic material ink is more preferably 1 nm or more and 180 nm or less, still more preferably 1 nm or more and 150 nm or less.

It is preferable that the blue cosmetic material ink in the present embodiment has negative skin irritation, that is, is highly safe for a living body. By making the skin irritation of the blue cosmetic material ink negative, the blue cosmetic material ink can be suitably used in purposes where the blue cosmetic material ink is in contact with the skin and the like while being used. Here, in the present specification, “negative skin irritation” means that the cell viability is more than 50% in a case where testing is performed using a three-dimensional skin model, which is an alternative method of skin irritation testing. In the alternative method of skin irritation testing, a 5% sodium dodecyl sulfate (SDS) solution is used as an irritant control and phosphate-buffered saline (PBS) is used as a negative control. The ink of interest is exposed to the three-dimensional skin model for 18 hours, and then the cell viability is assessed by the MTT test.

Examples of a method for making the skin irritation of the blue cosmetic material ink negative include a method in which all ingredients contained in the blue cosmetic material ink are selected from ingredients listed in the list of cosmetic ingredient display names based on the Pharmaceutical Affairs Law of Japan, ingredients that comply with the EU Cosmetics Directive (Cosmetics Directive 76/768/EEC), or ingredients listed in the International Cosmetic Ingredient Dictionary and Handbook (Jan. 1, 2002, 9th edition) by the US CTFA (Cosmetic, Toiletry & Fragrance Association, U.S.).

### 1-2. White Cosmetic Material Ink

Next, among the cosmetic material inks in the present embodiment, the white cosmetic material ink will be described in detail below.

### 1-2-1. Respective Ingredients of White Cosmetic Material Ink

The white cosmetic material ink in the present embodiment contains a white pigment (A2), acrylic particles (B), an alcohol-based solvent (C), and water (D). Furthermore, the white cosmetic material ink may contain ingredients other than these if necessary. Hereinafter, the respective ingredients will be described in detail.

### <White Pigment (A2)>

The white pigment (A2) contained in the white cosmetic material ink is not particularly limited as long as it is a pigment capable of producing a white color, but is preferably a pigment selected from ingredients listed in the list of cosmetic ingredient display names based on the Pharmaceutical Affairs Law of Japan, ingredients that comply with the EU Cosmetics Directive (Cosmetics Directive 76/768/EEC), or ingredients listed in the International Cosmetic Ingredient Dictionary and Handbook (Jan. 1, 2002, 9th edition) by the US CTFA (Cosmetic, Toiletry & Fragrance Association, U.S.) from the viewpoint of skin irritation. Specifically, the white pigment (A2) may be either of an inorganic pigment or an organic pigment used in arbitrary cosmetic products and the like known to those skilled in the art, but is preferably an inorganic pigment from the viewpoint of being suitably used without adversely affecting the skin.

Specifically, as such an inorganic pigment, the white pigment (A2) preferably includes at least one selected from titanium oxide, zinc oxide, cerium oxide, barium sulfate, talc, muscovite, phlogopite, red mica, biotite, synthetic mica, sericite, synthetic sericite, kaolin, silicon carbide, bentonite, smectite, silicic anhydride, aluminum oxide, magnesium oxide, zirconium oxide, antimony oxide, diatomaceous earth, aluminum silicate, magnesium aluminum metasilicate, calcium silicate, barium silicate, magnesium silicate, calcium carbonate, magnesium carbonate, hydroxyapatite, and boron nitride.

Among these, the white pigment (A2) more preferably includes titanium oxide from the viewpoints of water dispersion stability and color producing properties, safety, and biocompatibility as a cosmetic material.

The content of the white pigment (A2) is 4% by mass or more and 35% by mass or less with respect to the total amount of the white pigment (A2), acrylic particles (B), alcohol-based solvent (C), and water (D).

When the content of the white pigment (A2) is less than 4% by mass, it is difficult to favorably conceal discolored areas and the like with a cosmetic sheet produced using the white cosmetic material ink. When the content of the white pigment (A2) exceeds 35% by mass, it is difficult to stably eject the white cosmetic material ink from an inkjet printer.

The content of the white pigment (A2) is preferably 5% by mass or more, more preferably 7% by mass or more, still more preferably 10% by mass or more. The content of the white pigment (A2) is preferably 30% by mass or less, more preferably 20% by mass or less, still more preferably 15% by mass or less.

The white pigment (A2) has a median diameter (the median value (D50) of integrated values in particle size distribution) measured by a laser diffraction method of 300 nm or more and 900 nm or less. When the median diameter of the white pigment (A2) is less than 300 nm, it is difficult to conceal discolored areas and the like with a cosmetic sheet produced using the white cosmetic material ink. When the median diameter of the white pigment (A2) exceeds 900 nm, it is difficult to stably eject the white cosmetic material ink from an inkjet printer.

The median diameter of the white pigment (A2) is preferably 310 nm or more, more preferably 320 nm or more, still more preferably 340 nm or more. The median diameter of the white pigment (A2) is preferably 881 nm or less, more preferably 600 nm or less, still more preferably 500 nm or less.

### <Acrylic Particles (B)>

The white cosmetic material ink in the present embodiment contains acrylic particles (B). The acrylic particles (B) fix the white cosmetic material ink to the substrate and improve the water resistance and durability of the printed matter.

The acrylic particles (B) are not particularly limited as long as they are particles formed of a (meth)acrylic resin having the above-mentioned action. Specific examples of the acrylic particles (B) are the same as those in the case of the blue cosmetic material ink described above.

The content of the acrylic particles (B) is preferably 3% by mass or more, more preferably 3% by mass or more and 20% by mass or less with respect to the total amount of the white pigment (A2), acrylic particles (B), alcohol-based solvent (C), and water (D).

By setting the content of the acrylic particles (B) to 3% by mass or more, preferably 3% by mass or more and 20% by mass or less, not only the concealing properties of the white cosmetic material ink and the ejection stability in inkjet printing are improved but also the effect, such as excellent fixity (water resistance) in the printed matter, of the white cosmetic material ink can be obtained.

The content of the acrylic particles (B) is more preferably 4% by mass or more, still more preferably 6% by mass or more. The content of the acrylic particles (B) is more preferably 15% by mass or less, still more preferably 10% by mass or less.

The preferred range of the median diameter of the acrylic particles (B) and the effects obtained by setting the median diameter to be in such a range are the same as those in the case of the blue cosmetic material ink described above.

When the white cosmetic material ink is prepared, the acrylic particles (B) are mixed with the white pigment (A2), alcohol-based solvent (C), water (D) and the like usually in a state of being dispersed in a dispersion medium (slurry or dispersion state). The dispersion medium at this time is preferably a solvent that does not irritate the skin, and is preferably an alcohol-based solvent (C) or water (D) described below.

### <Alcohol-Based Solvent (C)>

The white cosmetic material ink in the present embodiment contains an alcohol-based solvent (C). The functions of the alcohol-based solvent (C) are the same as those in the case of the blue cosmetic material ink described above.

The preferred range of the number of carbon atoms in the alcohol-based solvent (C) and the effect obtained by setting the number of carbon atoms to be in such a range as well as the preferred properties of the alcohol-based solvent (C) are also the same as those in the case of the blue cosmetic material ink described above.

The preferred lower limit of the boiling point of the alcohol-based solvent (C) (or the boiling points of both the dihydric alcohol-based solvent (C1) and trihydric alcohol-based solvent (C2)) and the effects obtained by setting the boiling point to such a lower limit are the same as those in the case of the blue cosmetic material ink described above.

The white cosmetic material ink in the present embodiment also contains both the dihydric alcohol-based solvent (C1) and trihydric alcohol-based solvent (C2) as the alcohol-based solvent (C). The effects obtained by containing both of these are also the same as those in the case of the blue cosmetic material ink described above.

The effects obtained by containing the dihydric alcohol-based solvent (C1) as the alcohol-based solvent (C) as well as preferred specific examples of the dihydric alcohol-based solvent (C1) and the preferred action thereof are also the same as those in the case of the blue cosmetic material ink described above.

The effects obtained by containing the trihydric alcohol-based solvent (C2) as the alcohol-based solvent (C) as well as preferred specific examples of the trihydric alcohol-based solvent (C2) and the preferred action thereof are also the same as those in the case of the blue cosmetic material ink described above.

The total content of the dihydric alcohol-based solvent (C1) and trihydric alcohol-based solvent (C2) is 17% by mass or more and 55% by mass or less with respect to the total amount of the white pigment (A2), acrylic particles (B), alcohol-based solvent (C), and water (D).

When the total content of the dihydric alcohol-based solvent (C1) and trihydric alcohol-based solvent (C2) is in such a range, the viscosity of the white cosmetic material ink can be stably maintained in a range suitable for inkjet printing. When the viscosity of the white cosmetic material ink is in a range suitable for inkjet printing, the shapes of flying droplets after ink ejection can be stabilized, the droplet shapes when the droplets land on a substrate can be uniformized, and ink ejection deflection can be prevented.

When the total content of the dihydric alcohol-based solvent (C1) and trihydric alcohol-based solvent (C2) is in such a range, the area around the nozzles of an inkjet printer can be favorably moisturized. As a result, nozzle clogging and print defects due to drying are not caused, and the white cosmetic material ink can be stably ejected from an inkjet printer. Specifically, when the total content of the dihydric alcohol-based solvent (C1) and trihydric alcohol-based solvent (C2) exceeds 55% by mass, the pigment aggregates and this is likely to lead to ink ejection failure due to nozzle clogging.

The total content of the dihydric alcohol-based solvent (C1) and trihydric alcohol-based solvent (C2) is preferably 18% by mass or more, more preferably 20% by mass or more. The total content of the dihydric alcohol-based solvent (C1) and trihydric alcohol-based solvent (C2) is preferably 50% by mass or less, more preferably 40% by mass or less, still more preferably 30% by mass or less.

The content of the dihydric alcohol-based solvent (C1) is preferably 10% by mass or more and 40% by mass or less with respect to the total amount of the white pigment (A2), acrylic particles (B), alcohol-based solvent (C), and water (D). By setting the content of the dihydric alcohol-based solvent (C1) to be in such a range, it is easier to stably maintain the viscosity of the white cosmetic material ink in a range suitable for inkjet printing and the area around the nozzles of an inkjet printer can be more favorably moisturized. As a result, a white cosmetic material ink superior in ejection stability in inkjet printing can be obtained.

The content of the dihydric alcohol-based solvent (C1) is more preferably 40% by mass or less, still more preferably 20% by mass or less.

The content of the trihydric alcohol-based solvent (C2) is preferably 7% by mass or more and 45% by mass or less with respect to the total amount of the white pigment (A2), acrylic particles (B), alcohol-based solvent (C), and water (D). By setting the content of the trihydric alcohol-based solvent (C2) to be in such a range, it is easier to stably maintain the viscosity of the white cosmetic material ink in a range suitable for inkjet printing and the area around the nozzles of an inkjet printer can be more favorably moisturized. As a result, a white cosmetic material ink superior in ejection stability in inkjet printing can be obtained.

The content of the trihydric alcohol-based solvent (C2) is more preferably 8% by mass or more, still more preferably 10% by mass or more. The content of the trihydric alcohol-based solvent (C2) is more preferably 45% by mass or less, still more preferably 20% by mass or less.

### <Water (D)>

The white cosmetic material ink in the present embodiment contains water (D), preferably purified water. The functions and specific examples of water (D) are the same as those in the case of the blue cosmetic material ink described above.

The content of water (D) is preferably 30% by mass or more and 70% by mass or less, more preferably 55% by mass or more and 70% by mass or less with respect to the total amount of the white pigment (A2), acrylic particles (B), alcohol-based solvent (C), and water (D). By setting the content of water (D) to be in such a range, the viscosity of the white cosmetic material ink can be likely to fall in a range suitable for inkjet printing.

### <Other Ingredients>

The white cosmetic material ink may further contain various kinds of arbitrary additives and the like known in the art in a range in which the effects, such as concealing properties and the ejection stability in inkjet printing, of the white cosmetic material ink in the present embodiment are not impaired. Specific examples of the various kinds of arbitrary additives are the same as those in the case of the blue cosmetic material ink described above.

### 1-2-2. Method for Preparing White Cosmetic Material Ink

The white cosmetic material ink in the present embodiment can be prepared by, for example, mixing a slurry or dispersion containing the white pigment (A2), acrylic particles (B), alcohol-based solvent (C), and water (D) and, if necessary, other ingredients using a disperser, or the like. The method for mixing the respective ingredients is the same as that in the case of the blue cosmetic material ink described above.

### 1-2-3. Physical Properties of White Cosmetic Material Ink

It is preferable that the white cosmetic material ink in the present embodiment has a viscosity of 3 mPa·s or more and 20 mPa·s or less at 25° C. when the viscosity is measured at a shear rate of 1000 (1/s) using a cone-plate viscometer. As the viscosity of the white cosmetic material ink is in such a range, the white cosmetic material ink can be more stably ejected from an inkjet printer. Specifically, the shapes of flying droplets after ink ejection can be stabilized, the droplet shapes when the droplets land on a substrate can be uniformized, and ink ejection deflection can be prevented.

The viscosity of the white cosmetic material ink when measured under the same conditions as above is more preferably 4 mPa·s or more, still more preferably 5 mPa·s or more. The viscosity of the white cosmetic material ink when measured under the same conditions as above is more preferably 10 mPa·s or less, still more preferably 8 mPa·s or less.

The preferred pH range of the white cosmetic material ink and the effects obtained by setting the pH to be in such a range are the same as those in the case of the blue cosmetic material ink described above.

The preferred range of the surface tension of the white cosmetic material ink at 25° C. and the effects obtained by setting the surface tension to be in such a range are also the same as those in the case of the blue cosmetic material ink described above.

The preferred range of the median diameter (the median value (D50) of integrated values in particle size distribution) of the particles contained in the white cosmetic material ink and the 90% value (D90) of the integrated values in the particle size distribution as well as the effects obtained by setting the median diameter and the 90% value to be in such ranges are the same as those in the case of the blue cosmetic material ink described above.

It is preferable that the white cosmetic material ink in the present embodiment also has negative skin irritation, that is, is highly safe for a living body. Details of the negative skin irritation of the white cosmetic material ink are the same as those in the case of the blue cosmetic material inks described above.

### 2. Ink Cartridge

In a case where the blue or white cosmetic material ink in the above-described embodiment is used as an ink for inkjet printing, the blue or white cosmetic material ink may be filled and stored in a cartridge, for example. The type and/or structure of the cartridge is not particularly limited, and may be appropriately selected to suit the printer for applying the cosmetic material ink by printing. An example of such an ink cartridge includes a cartridge including an ink housing section for housing the blue or white cosmetic material ink and an ink supply port for supplying the blue or white cosmetic material ink to a recording nozzle of a printer.

### 3. Cosmetic Sheet

### 3-1. Cosmetic Sheet Using Cosmetic Material Ink (Blue Cosmetic Material Ink or White Cosmetic Material Ink)

The cosmetic sheet in the present embodiment includes a substrate and a coating film that is formed on the substrate and has an ink layer formed of the blue or white cosmetic material ink in the above-described embodiment.

Such a cosmetic sheet can be produced by, for example, a method including a step of applying the cosmetic material ink in the above-described embodiment to a substrate by printing and a step of drying the cosmetic material ink applied to the substrate.

The method for applying the cosmetic material ink to a substrate by printing is not particularly limited, and examples thereof include inkjet printing, screen printing, offset printing, and gravure printing. Among these, it is preferable to employ inkjet printing from the viewpoint that the cosmetic material ink in the above-described embodiment is excellent in ejection stability in inkjet printing. According to inkjet printing, the blue or white cosmetic material ink can be applied to the substrate a plurality of times depending on the skin condition, skin color, and the like of the user of the cosmetic sheet.

Hereinafter, a method for producing a cosmetic sheet will be described in more detail, taking a case where the cosmetic material ink is applied by inkjet printing as an example, but the method for producing the cosmetic sheet is not limited to this method.

In a case where the cosmetic material ink is applied to a substrate by inkjet printing, the inkjet printer is not particularly limited as long as it is an arbitrary apparatus known to those skilled in the art. For example, any of a piezoelectric apparatus, a thermal apparatus, or an electrostatic apparatus can be used. Among these, an inkjet printer of a piezoelectric element type is preferable from the viewpoint that heating is not required as in the thermal inkjet type.

The cosmetic material ink may be applied to the substrate only one time or two or more times. In a case where the cosmetic material ink is applied to a substrate a plurality of times, drying may be performed every time the cosmetic material ink is applied, or drying may be performed after the cosmetic material ink is applied the plurality of times.

The method for drying the cosmetic material ink applied to the substrate is not particularly limited as long as it is a method capable of removing the alcohol-based solvent (C) and water (D) in the cosmetic material ink. The drying method may be, for example, a method in which drying is performed at room temperature in the atmospheric pressure or a method in which heating to a predetermined temperature and/or pressure reduction are performed for drying. In the case of performing heating, it is preferable to perform heating to, for example, 25° C. to 50° C. In this temperature range, drying can be efficiently performed without deteriorating the sheet and the solids in the cosmetic material ink. Meanwhile, in the case pf performing pressure reduction, it is preferable to reduce the pressure to −0.1 to 0 MPa. By reducing the pressure in this range, the ink can be efficiently dried.

The substrate to which the cosmetic material ink is applied is not particularly limited as long as it does not impair the suitable properties of the cosmetic material ink in the above-described embodiment (favorable skin color expressiveness of the blue cosmetic material ink or favorable concealing properties of the white cosmetic material ink) and the effect such as ejection stability in inkjet printing, and one formed of the same material as the base material of an arbitrary cosmetic sheet known to those skilled in the art can be used as the substrate. Examples of such a substrate include plain paper, special purpose recording paper, plastic, and cloth. The substrate may have only one layer or two or more laminated layers. Among these, a sheet that is formed of a biocompatible material and is directly or indirectly affixed to the skin or is brought into close contact with the skin for use is particularly preferable.

Examples of the biocompatible material include polyesters represented by polyglycolic acid, polylactic acid, polycaprolactone, polyethylene succinate, polyethylene terephthalate, or copolymers thereof; polyethers represented by polyethylene glycol and polypropylene glycol; polyamides represented by nylon, polyglutamic acid, polyaspartic acid, or salts thereof; polysaccharides represented by pullulan, cellulose, starch, chitin, chitosan, alginic acid, hyaluronic acid, and cornstarch, or salts thereof; silicones represented by acrylic silicones and trimethylsiloxysilicates; acrylic acids represented by alkyl acrylates, silicone acrylates, acrylic acid amides, and copolymers thereof; polyvinyl alcohol; polyurethane; polycarbonates; polyanhydrides; polyethylene; polypropylene; porous layer coating sheets, and nanofiber sheets. Examples of sheets formed of such biocompatible materials include sheets for fixing medical members, sports taping sheets, skin decoration sheets, cosmetic sheets, and surgical adhesion barriers.

The thickness of the substrate is not particularly limited, and can be appropriately selected depending on the purpose of the cosmetic sheet, the material of the substrate, and the like. The substrate is preferably a sheet having a thickness of, for example, 10 nm or more and 3000 nm or less.

In a case where an image is formed on such a thin film sheet, printing is performed on a laminate in which a thin film sheet that is the substrate and a support that supports the thin film sheet are laminated, and the thin film may be peeled off from the support after printing. Such a support is not particularly limited, but can be formed of, for example, a highly water-absorbable material. In a case where the support is water-absorbable, the alcohol-based solvent (C) and water (D) can be quickly absorbed into the support and only the solids in the cosmetic material ink in the above-described embodiment can be fixed to the surface of the thin film sheet that is the substrate. Therefore, bleeding and the like are less likely to occur in the obtained image, and high-definition image formation is possible. Examples of the highly water-absorbable support include base materials formed of paper, cloth, a nonwoven fabric, a textile, a porous layer coated sheet, a nanofiber sheet, a water-absorbent polymer, a water-soluble polymer, and the like.

The cosmetic sheet produced using the blue cosmetic material ink in the above-described embodiment can express favorable brightness in its coating film. For this reason, the cosmetic sheet produced using the blue cosmetic material ink can be used affixed to the skin and the like for use, and can be used, for example, for makeup purposes, special effects makeup purposes, face or body painting purposes, bar code purposes, and other display purposes.

Alternatively, the cosmetic sheet produced using the white cosmetic material ink in the above-described embodiment is excellent in concealing properties for discolored areas. Hence, the cosmetic sheet produced using the white cosmetic material ink, for example, is affixed to the skin for use, and can be suitably used for medical purposes to make discolored areas less noticeable.

### 3-2. Cosmetic Sheet Produced Using Cosmetic Material Ink and Cosmetic Material Inks of Other Colors

The cosmetic sheet in the present embodiment may include a substrate and a coating film that is formed on the substrate and has an ink layer formed of the blue or white cosmetic material ink in the above-described embodiment and one or more ink layers formed of cosmetic material inks of other colors.

Such a cosmetic sheet can be produced by, for example, a method including a step of applying (preferably at different positions) droplets of the blue cosmetic material ink (or white cosmetic material ink) in the above-described embodiment and droplets of cosmetic material inks of one or more colors selected from the group consisting of a white cosmetic material ink (or blue cosmetic material ink), a red cosmetic material ink, a yellow cosmetic material ink, and a black cosmetic material ink and a step of drying the cosmetic material inks applied to the substrate.

By applying the cosmetic material inks of the respective colors at different positions, a coating film having the desired brightness and spectral reflectance can be easily formed in the desired area of a substrate. For example, a coating film that expresses skin color of the desired tone, a coating film having a spectral reflectance that can be recognized as skin color, and the like can be formed in the desired area of a substrate.

In the case of using cosmetic material inks of other colors, it is preferable to use cosmetic material inks of two or more colors selected from the group consisting of a white cosmetic material ink (or blue cosmetic material ink), a red cosmetic material ink, a yellow cosmetic material ink, and a black cosmetic material ink. It is more preferable to use cosmetic material inks of three colors of a white cosmetic material ink (or blue cosmetic material ink), a red cosmetic material ink, and a yellow cosmetic material ink. By combining the blue cosmetic material ink or white cosmetic material ink in the above-described embodiment with cosmetic material inks of these colors, for example, it becomes easier to form a coating film that expresses skin color of the desired tone and a coating film having a spectral reflectance that can be sensorily recognized as skin color, and a cosmetic sheet can be produced on which a coating film that exhibits high concealing properties for discolored areas and the like is formed.

By appropriately adjusting the amounts of these cosmetic material inks of the respective colors applied and/or the area to be coated depending on the purpose of the cosmetic sheet as well, it is possible to form a high-definition coating film having the desired brightness and spectral reflectance in the desired area of a substrate.

The compositions of a white cosmetic material ink or a blue cosmetic material ink, a red cosmetic material ink, a yellow cosmetic material ink, and a black cosmetic material ink as the cosmetic material inks of other colors are not particularly limited, but it is preferable that the cosmetic material inks contain ingredients roughly similar to those of the blue cosmetic material ink or white cosmetic material ink in the above-described embodiment. For example, it is preferable to contain a pigment other than a blue pigment (or a pigment other than a white pigment), acrylic particles (B), an alcohol-based solvent (C), and water (D).

The method for applying the cosmetic material inks of the respective colors is not particularly limited, and examples thereof include inkjet printing, screen printing, offset printing, and gravure printing. Among these, inkjet printing is preferable from the viewpoint that the blue cosmetic material ink and the white cosmetic material ink in the above-described embodiments are excellent in ejection stability in inkjet printing.

In the case of forming a coating film by inkjet printing, first, the blue cosmetic material ink (or white cosmetic material ink) and cosmetic material inks of one or more colors selected from the group consisting of a white cosmetic material ink (or blue cosmetic material ink), a red cosmetic material ink, a yellow cosmetic material ink, and a black cosmetic material ink are filled in the ink tanks of an inkjet printer, respectively. Then, for example, the cosmetic material inks of the respective colors are applied to the same position or different positions.

Specifically, in the case of applying the cosmetic material inks of the respective colors to the same position, printing for the first color is performed using an inkjet printer and then printing for the second color is performed without moving or rotating the nozzle or the substrate. Printing for the third color and onwards is also performed in the same manner. Meanwhile, in the case of applying the cosmetic material inks of the respective colors to different positions, printing for the first color is performed using an inkjet printer, then the nozzle or the substrate is moved or rotated in a certain direction, and printing for the second color is performed. Subsequently, at the time of printing for the third color and onwards as well, the nozzle or the substrate is moved or rotated in a certain direction and then printing is performed. This makes it possible to cause droplets of cosmetic material inks of the respective colors to properly land at different positions. From the viewpoint that the coating film on the substrate becomes more uniform and a high-definition coating film is formed, it is preferable to apply the cosmetic material inks of the respective colors at different positions.

The substrate to which the ink is applied is the same as that in the case of producing a cosmetic sheet using only the blue or white cosmetic material ink described above.

The cosmetic sheet produced using the blue cosmetic material ink in the above-described embodiment and cosmetic material inks of other colors can favorably express skin color in its coating film. The cosmetic sheet produced using the white cosmetic material ink in the above-described embodiment and cosmetic material inks of other colors is not only excellent in concealing properties for discolored areas but can also form a coating film that expresses skin color of the desired tone, a coating film having a spectral reflectance that can be recognized as skin color, and the like. Hence, the cosmetic sheet, for example, is affixed to the skin for use, and can be used not only for medical purposes to make discolored areas and the like less noticeable, but also for various cosmetic purposes such as skin coloring, beautification, and makeup.

The outline of the present invention has been described above, and the cosmetic material ink and cosmetic sheet in the present embodiment are summarized as follows.

(1) A cosmetic material ink according to a first aspect of the present embodiment is a blue cosmetic material ink comprising:
a blue pigment (A1);
acrylic particles (B);
an alcohol-based solvent (C); and
water (D),
wherein a content of the blue pigment (A1) is 0.5% by mass or more and 4.5% by mass or less with respect to a total amount of the blue pigment (A1), the acrylic particles (B), the alcohol-based solvent (C), and the water (D),
the alcohol-based solvent (C) includes a dihydric alcohol-based solvent (C1) and a trihydric alcohol-based solvent (C2), and
a total content of the dihydric alcohol-based solvent (C1) and the trihydric alcohol-based solvent (C2) is 30% by mass or more and 85% by mass or less with respect to the total amount of the blue pigment (A1), the acrylic particles (B), the alcohol-based solvent (C), and the water (D).

(2) In the cosmetic material ink according to (1), it is preferable that a content of the dihydric alcohol-based solvent (C1) is 15% by mass or more and 45% by mass or less and a content of the trihydric alcohol-based solvent (C2) is 15% by mass or more and 45% by mass or less with respect to the total amount of the blue pigment (A1), the acrylic particles (B), the alcohol-based solvent (C), and the water (D).

(3) In the cosmetic material ink according to (1) or (2), it is more preferable that the blue pigment (A1) includes at least one selected from Prussian blue (ferric ferrocyanide), ultramarine blue, lapis lazuli, blue verditer, aluminum-cobalt oxide, aluminum-zinc-cobalt oxide, silicon-cobalt oxide, silicon-zinc-cobalt oxide, cobalt pigment, smalt, cobalt blue, cobalt stannate, cobalt chrome blue, cobalt-aluminum-silicon oxide, and manganese blue.

(4) In the cosmetic material ink according to any one of (1) to (3), it is still more preferable that the blue pigment (A1) has a median diameter of 90 nm or more and 500 nm or less.

(5) In the cosmetic material ink according to any one of (1) to (4), it is particularly preferable that the cosmetic material ink has a viscosity of 3 mPa·s or more and 15 mPa·s or less at 25° C. when the viscosity is measured at a shear rate of 1000 (1/s) using a cone-plate viscometer.

(6) A cosmetic material ink according to another first aspect of the present embodiment is a white cosmetic material ink comprising:
a white pigment (A2);
acrylic particles (B);
an alcohol-based solvent (C); and
water (D),
wherein the white pigment (A2) has a median diameter of 300 nm or more and 900 nm or less,
a content of the white pigment (A2) is 4% by mass or more and 35% by mass or less with respect to a total amount of the white pigment (A2), the acrylic particles (B), the alcohol-based solvent (C), and the water (D),
the alcohol-based solvent (C) includes a dihydric alcohol-based solvent (C1) and a trihydric alcohol-based solvent (C2), and
a total content of the dihydric alcohol-based solvent (C1) and the trihydric alcohol-based solvent (C2) is 17% by mass or more and 55% by mass or less with respect to the total amount of the white pigment (A2), the acrylic particles (B), the alcohol-based solvent (C), and the water (D).

(7) In the cosmetic material ink according to (6), it is preferable that a content of the acrylic particles (B) is 3% by mass or more and 20% by mass or less with respect to the total amount of the white pigment (A2), the acrylic particles (B), the alcohol-based solvent (C), and the water (D).

(8) In the cosmetic material ink according to (6) or (7), it is more preferable that the cosmetic material ink has a viscosity of 3 mPa·s or more and 20 mPa·s or less at 25° C. when the viscosity is measured at a shear rate of 1000 (1/s) using a cone-plate viscometer.

(9) In the cosmetic material ink according to any one of (6) to (8), it is still more preferable that a content of the dihydric alcohol-based solvent (C1) is 10% by mass or more and 40% by mass or less and a content of the trihydric alcohol-based solvent (C2) is 7% by mass or more and 45% by mass or less with respect to the total amount of the white pigment (A2), the acrylic particles (B), the alcohol-based solvent (C), and the water (D).

(10) In the cosmetic material ink according to any one of (6) to (9), it is particularly preferable that the white pigment (A2) includes at least one selected from titanium oxide, zinc oxide, cerium oxide, barium sulfate, talc, muscovite, phlogopite, red mica, biotite, synthetic mica, sericite, synthetic sericite, kaolin, silicon carbide, bentonite, smectite, silicic anhydride, aluminum oxide, magnesium oxide, zirconium oxide, antimony oxide, diatomaceous earth, aluminum silicate, magnesium aluminum metasilicate, calcium silicate, barium silicate, magnesium silicate, calcium carbonate, magnesium carbonate, hydroxyapatite, and boron nitride.

(11) In the cosmetic material ink according to any one of (1) to (10), it is preferable that the alcohol-based solvent (C) includes an alcohol-based solvent having a boiling point of 200° C. or more.

(12) In the cosmetic material ink according to any one of (1) to (11), it is more preferable that the dihydric alcohol-based solvent (C1) contains at least one selected from diethylene glycol, propylene glycol, 1,3-propanediol, butylene glycol, and hexanediol.

(13) In the cosmetic material ink according to any one of (1) to (12), it is still more preferable that the acrylic particles (B) contain at least one of a homopolymer of a (meth)acrylic monomer or a copolymer of two and more kinds of (meth)acrylic monomers.

(14) In the cosmetic material ink according to any one of (1) to (13), it is particularly preferable that the cosmetic material ink is an ink for inkjet printing to be applied to a sheet having a thickness of 10 nm or more and 3000 nm or less by inkjet printing.

(15) A cosmetic sheet according to a second aspect of the present embodiment comprises:
a sheet having a thickness of 10 nm or more and 3000 nm or less; and
a coating film that is formed on the sheet and has at least an ink layer formed of the cosmetic material ink according to any one of (1) to (14).

### EXAMPLES

Hereinafter, the present invention will be described more specifically with reference to Examples, but the present invention is not limited to Examples in any way.

### [Materials of Blue Cosmetic Material Ink]

The materials of the blue cosmetic material ink used in each of Examples and Comparative Examples are as follows. The median diameter of each particle is the median value (D50) of integrated values in the particle size distribution measured by a laser diffraction method.
Blue pigment (A1): Prussian blue (median diameter: 105 nm)
Acrylic particles (B): Acrylates copolymer (median diameter: 5 nm)
Dihydric alcohol-based solvent (C1): 1,3-Propanediol
Trihydric alcohol-based solvent (C2): Glycerin
Water (D): Purified water

### Example 1

In Example 1, each blue cosmetic material ink was prepared by varying the content of the blue pigment (A1) contained in the blue cosmetic material ink. Specifically, the respective blue cosmetic material inks were prepared by mixing the respective materials mentioned above at the ingredient ratios in Examples 1-1 to 1-4 and Comparative Examples 1-1 and 1-2 presented in Table 1 below.

### [Evaluation]

The viscosity of each of the prepared blue cosmetic material inks was measured by the method described below. Furthermore, the skin color expressiveness and ejection stability in inkjet printing of each of the blue cosmetic material inks were also evaluated by the methods described below.

### (Method for Measuring Viscosity of Blue Cosmetic Material Ink)

The viscosity of the blue cosmetic material ink was measured using a cone-plate viscometer (viscosity/viscoelasticity measuring instrument HAAKE MARS manufactured by Thermo Fisher Scientific Inc.). Specifically, 0.3 cc of blue cosmetic material ink was taken with a syringe and sampled on a cone-plate, and the measurement was performed at a temperature of 25° C. while the shear rate was changed from 0.1 to 1000 (1/s).

### (Method for Evaluating Skin Color Expressiveness of Blue Cosmetic Material Ink)

With regard to the skin color expressiveness of the blue cosmetic material ink, a cosmetic sheet was produced using the blue cosmetic material ink of each of Examples and Comparative Examples and the evaluation was performed using the sheet. First, each of the prepared blue cosmetic material inks was filled in an ink tank of an inkjet printer equipped with an LB3 inkjet nozzle (manufactured by Panasonic Precision Devices Co., Ltd.). A polylactic acid sheet having a thickness of 400 nm was attached to a support formed of filter paper, and this was prepared as a substrate. Next, the blue cosmetic material ink was applied onto the polylactic acid sheet using an inkjet printer to form a circular pattern (fill pattern) having a radius of 30 mm. At this time, the amount of blue cosmetic material ink applied was 10 nl. Furthermore, each of a red cosmetic material ink and a yellow cosmetic material ink was laminated on the pattern of blue cosmetic material ink by 100 nl as a fill pattern by the same method. The sheet after printing was dried in an environment of 50° C. to obtain each cosmetic sheet.

Next, the skin color expressiveness of the coating film formed on the obtained cosmetic sheet was evaluated by the following method. Specifically, the L* value, a* value and b* value in the L*a*b* color system of the obtained coating film were measured using a spectrophotometer (“CM-700d” manufactured by Konica Minolta, Inc.) under the conditions of main light source: D50 and measurement mode: SCE. Meanwhile, the L* value, a* value, and b* value in the L*a*b* color system of a skin model (BIOSKIN (Product Model Number: BIO manufactured by Beaulax Co., Ltd.)) were also measured in the same manner. Then, the color difference ΔE*ab between the coating film and the skin model was calculated. In a case where ΔE*ab was less than 13.0, it was determined that the coating film exhibited suitable skin color expressiveness (denoted as “A” in Tables 1 and 2 below). Meanwhile, in a case where ΔE*ab was 13.0 or more, it was determined that the coating film did not exhibit suitable skin color expressiveness (denoted as “C” in Tables 1 and 2 below).

### (Method for Evaluating Ejection Stability in Inkjet Printing)

The ejection stability of the blue cosmetic material ink in inkjet printing was measured by the following method. First, the prepared blue cosmetic material ink was filled in an ink tank of an inkjet printer equipped with an LB3 inkjet nozzle (manufactured by Panasonic Precision Devices Co., Ltd.). Then, the flight of droplets of the blue cosmetic material ink ejected from the inkjet head was observed and photographed on a camera monitor using an inkjet ejection observing apparatus. The flight angle of the ink droplets with respect to the inkjet nozzle was measured from the observed and photographed images and videos, and evaluated as the ejection deflection (angle) in inkjet printing. In a case where the ejection deflection was less than 2°, it was determined that stable ejection was possible.

TABLE 1

| | | | Compar- | | | | | Compar- |
|---|---|---|---|---|---|---|---|---|
| | | | ative | | | | | ative |
| | | | Example | Example | Example | Example | Example | Example |
| | | | 1-1 | 1-1 | 1-2 | 1-3 | 1-4 | 1-2 |
| Ingredients | Blue | Content (% by mass) | 0 | 0.52 | 1.04 | 2.59 | 4.15 | 5.18 |
| of blue | pigment (A1) | (A1)/{(A1) + (B) + (C) + (D)} × 100 | | | | | | |
| cosmetic | Acrylic | Content (% by mass) | 2.60 | 2.60 | 2.60 | 2.60 | 2.60 | 2.60 |
| material | particles (B) | (B)/{(A1) + (B) + (C) + (D)} × 100 | | | | | | |
| ink | Alcohol-based | Content (% by mass) | 41.5 | 41.5 | 41.5 | 41.5 | 41.5 | 41.5 |
| | solvent (C) | {(C1) + (C2)}/{(A1) + (B) + (C) + (D)} × 100 | | | | | | |
| | Dihydric | Content (% by mass) | 20.7 | 20.7 | 20.7 | 20.7 | 20.7 | 20.7 |
| | alcohol-based | (C1)/{(A1) + (B) + (C) + (D)} × 100 | | | | | | |
| | solvent (C1) | | | | | | | |
| | Trihydric | Content (% by mass) | 20.7 | 20.7 | 20.7 | 20.7 | 20.7 | 20.7 |
| | alcohol-based | (C2)/{(A1) + (B) + (C) + (D)} × 100 | | | | | | |
| | solvent (C2) | | | | | | | |
| | Water (D) | Content (% by mass) | 55.90 | 55.38 | 54.86 | 53.31 | 51.75 | 50.72 |
| | | (D)/{(A1) + (B) + (C) + (D)} × 100 | | | | | | |
| Physical | Viscosity (mPa · s) | | 5.4 | 5.4 | 5.4 | 5.4 | 5.4 | 5.4 |
| properties | | | | | | | | |

| | | | | | | | |
|---|---|---|---|---|---|---|---|
| Evaluation | Skin color expressiveness | C | A | A | A | A | C |
| | Ejection deflection/flight angle of ink droplet | Less | Less | Less | Less | Less | Less |

| | | | | | |
|---|---|---|---|---|---|
| than 2° | than 2° | than 2° | than 2° | than 2° | than 2° |

### (Results)

As presented in Table 1 above, when the content of the blue pigment (A1) contained in the blue cosmetic material ink was set to be in the range of 0.5% by mass or more and 4.5% by mass or less with respect to the total amount of the blue pigment (A1), acrylic particles (B), alcohol-based solvent (C), and water (D), it was possible to improve the skin color expressiveness of printed matter obtained by using the blue cosmetic material ink (see Examples 1-1 to 1-4).

On the other hand, it was not possible to improve the skin color expressiveness when the content of the blue pigment (A1) in the blue cosmetic material ink was set to 0% by mass (see Comparative Example 1-1). Furthermore, it was not possible to improve the skin color expressiveness when the content of the blue pigment (A1) in the blue cosmetic material ink was increased to 5.18% by mass (see Comparative Example 1-2).

In all of the blue cosmetic material inks of Examples 1-1 to 1-4 and Comparative Examples 1-1 and 1-2 as well, the ejection stability in inkjet printing was favorable.

### Example 2

In Example 2, each blue cosmetic material ink was prepared by varying the contents of the dihydric alcohol-based solvent (C1) and trihydric alcohol-based solvent (C2) contained in the blue cosmetic material ink. Specifically, the respective blue cosmetic material inks were prepared by mixing the respective materials mentioned above at the ingredient ratios in Examples 2-1 to 2-3 and Comparative Examples 2-1 to 2-5 presented in Table 2 below.

### [Evaluation]

The viscosity of each of the prepared blue cosmetic material inks was measured by the same method as in Example 1. Furthermore, the skin color expressiveness and ejection stability in inkjet printing of each of the blue cosmetic material inks were also evaluated by the same methods as in Example 1.

TABLE 2

| | | | Comparative | Comparative | Comparative | Comparative |
|---|---|---|---|---|---|---|
| | | | Example | Example | Example | Example |
| | | | 2-1 | 2-2 | 2-3 | 2-4 |
| Ingredients | Blue | Content (% by mass) | 2.59 | 2.59 | 2.59 | 2.59 |
| of blue | pigment (A1) | (A1)/{(A1) + (B) + (C) + (D)} × 100 | | | | |
| cosmetic | Acrylic | Content (% by mass) | 2.60 | 2.60 | 2.60 | 2.60 |
| material | particles (B) | (B)/{(A1) + (B) + (C) + (D)} × 100 | | | | |
| ink | Alcohol-based | Content (% by mass) | 20.7 | 20.7 | 41.5 | 20.7 |
| | solvent (C) | {(C1) + (C2)}/{(A1) + (B) + (C) + (D)} × 100 | | | | |
| | Dihydric | Content (% by mass) | 10.4 | 0.0 | 0.0 | 20.7 |
| | alcohol-based | (C1)/{(A1) + (B) + (C) + (D)} × 100 | | | | |
| | solvent (C1) | | | | | |
| | Trihydric | Content (% by mass) | 10.4 | 20.7 | 41.5 | 0.0 |
| | alcohol-based | (C2)/{(A1) + (B) + (C) + (D)} × 100 | | | | |
| | solvent (C2) | | | | | |
| | Water (D) | Content (% by mass) | 74.11 | 74.11 | 53.31 | 74.11 |
| | | (D)/{(A1) + (B) + (C) + (D)} × 100 | | | | |
| Physical | Viscosity (mPa · s) | | 3 | 3 | 9 | 3 |
| properties | | | | | | |

| | | | | | |
|---|---|---|---|---|---|
| Evaluation | Skin color expressiveness | A | A | A | A |
| | Ejection deflection/flight angle of ink droplet | 3° to | 3° to | 2° to | 3° to |

| | | | |
|---|---|---|---|
| 5° | 5° | 5° | 5° |

| | | | | Example | | |
|---|---|---|---|---|---|---|
| | | | Comparative | 2-1 | | |
| | | | Example | (Example | Example | Example |
| | | | 2-5 | 1-3) | 2-2 | 2-3 |
| Ingredients | Blue | Content (% by mass) | 2.59 | 2.59 | 2.59 | 2.59 |
| of blue | pigment (A1) | (A1)/{(A1) + (B) + (C) + (D)} × 100 | | | | |
| cosmetic | Acrylic | Content (% by mass) | 2.60 | 2.60 | 2.60 | 2.60 |
| material | particles (B) | (B)/{(A1) + (B) + (C) + (D)} × 100 | | | | |
| ink | Alcohol-based | Content (% by mass) | 41.5 | 41.5 | 62.2 | 82.9 |
| | solvent (C) | {(C1) + (C2)}/{(A1) + (B) + (C) + (D)} × 100 | | | | |
| | Dihydric | Content (% by mass) | 41.5 | 20.7 | 31.1 | 41.5 |
| | alcohol-based | (C1)/{(A1) + (B) + (C) + (D)} × 100 | | | | |
| | solvent (C1) | | | | | |
| | Trihydric | Content (% by mass) | 0.0 | 20.7 | 31.1 | 41.5 |
| | alcohol-based | (C2)/{(A1) + (B) + (C) + (D)} × 100 | | | | |
| | solvent (C2) | | | | | |
| | Water (D) | Content (% by mass) | 53.31 | 53.31 | 32.61 | 11.91 |
| | | (D)/{(A1) + (B) + (C) + (D)} × 100 | | | | |
| Physical | Viscosity (mPa · s) | | 9 | 5.4 | 7.7 | 10 |
| properties | | | | | | |

| | | | | | |
|---|---|---|---|---|---|
| Evaluation | Skin color expressiveness | A | A | A | A |
| | Ejection deflection/flight angle of ink droplet | 2° to | Less | Less | Less |

| | | | |
|---|---|---|---|
| 5° | than 2° | than 2° | than 2° |

### (Results)

As presented in Table 2 above, it was possible to improve the ejection stability in inkjet printing when both the dihydric alcohol-based solvent (C1) and the trihydric alcohol-based solvent (C2) were contained as the alcohol-based solvent (C) and the content of the alcohol-based solvent (C) was set to 30% by mass or more and 85% by mass or less with respect to the total amount of the blue pigment (A1), acrylic particles (B), alcohol-based solvent (C), and water (D) (see Examples 2-1 to 2-3).

On the other hand, the ejection stability in inkjet printing was deteriorated when the content of the alcohol-based solvent (C) was decreased to 20.7% by mass (see Comparative Examples 2-1, 2-2, and 2-4). Furthermore, the ejection stability in inkjet printing was deteriorated in a case where both the dihydric alcohol-based solvent (C1) and trihydric alcohol-based solvent (C2) were not contained as the alcohol-based solvent (C) as well (see Comparative Examples 2-3 and 2-5).

In all of the blue cosmetic material inks of Examples 2-1 to 2-3 and Comparative Examples 2-1 to 2-5 as well, the skin color expressiveness was favorable.

### [Materials of White Cosmetic Material Ink]

The materials of the white cosmetic material ink used in each of Examples and Comparative Examples are as follows. The median diameter of each particle is the median value (D50) of integrated values in the particle size distribution measured by a laser diffraction method, as in the case of the blue cosmetic material ink.
White pigment (A2): Titanium dioxide (median diameter: 175 nm, 250 nm, 340 nm, 881 nm and 950 nm)
Acrylic particles (B): Acrylates copolymer (median diameter: 5 nm)
Dihydric alcohol-based solvent (C1): 1,3-Propanediol
Trihydric alcohol-based solvent (C2): Glycerin
Water (D): Purified water

### Example 3

In Example 3, each white cosmetic material ink was prepared by changing the median diameter of the white pigment (A2) contained in the white cosmetic material ink. Specifically, the respective white cosmetic material inks were prepared by mixing the respective materials mentioned above at the respective ingredient ratios using the white pigment (A2) having the median diameter in Examples 3-1 and 3-2 and Comparative Examples 3-1 to 3-3 presented in Table 3 below.

### [Evaluation]

The viscosity of each of the prepared white cosmetic material inks was measured by the same method as in the case of the blue cosmetic material inks described above. Furthermore, the ejection stability of each white cosmetic material ink in inkjet printing and the concealing properties of the cosmetic sheets produced using the respective white cosmetic material inks for discolored areas were evaluated by the methods described below. In addition, as reference evaluation, the fixity (water resistance) of each white cosmetic material ink was also evaluated by the method described below although fixity is not related to the effects (concealing properties and ejection stability in inkjet printing) of the white cosmetic material ink according to the present invention.

### (Method for Evaluating Ejection Stability in Inkjet Printing)

The ejection stability of the white cosmetic material ink in inkjet printing was measured by the following method. First, each of the prepared white cosmetic material inks was filled in an ink tank of an inkjet printer equipped with an LB3 inkjet nozzle (manufactured by Panasonic Precision Devices Co., Ltd.). Then, a circular pattern (fill pattern) having a radius of 30 mm was formed on an OHP sheet (transmittance of light having a wavelength of 380 to 780 nm: 90%, OHP film (Product Model Number: 27077) manufactured by A-one) that was a substrate using an inkjet printer. At this time, the amount of the white cosmetic material ink applied was 10 ul. The sheet after printing was dried in an environment of 50° C. to obtain each printed matter. Thereafter, the presence or absence of nozzle clogging in the inkjet printer was visually examined, and the print pattern was also carefully visually observed.

The evaluation was performed according to the following criteria.
AA: There is no nozzle clogging and no print defects.
A: There are no obvious print defects, but the print directionality is visible when carefully observed (the print pattern is not a uniform print pattern but the direction in which the ink droplets are aligned can be visually recognized).
C: Nozzle clogging occurs and streaky print defects are also generated.

### (Method for Evaluating Concealing Properties for Discolored Area)

With regard to the concealing properties for discolored areas, a cosmetic sheet was produced using the white cosmetic material ink of each of Examples and Comparative Examples and the evaluation was performed using the sheet. First, each of the prepared white cosmetic material inks was filled in an ink tank of an inkjet printer equipped with an LB3 inkjet nozzle (manufactured by Panasonic Precision Devices Co., Ltd.). A polylactic acid sheet having a thickness of 400 nm was attached to a support formed of filter paper, and this was prepared as a substrate. Next, the white cosmetic material ink was applied onto the polylactic acid sheet using an inkjet printer to form a circular pattern (fill pattern) having a radius of 30 mm. At this time, the amount of each cosmetic material ink applied was 10 ul. The sheet after printing was dried in an environment of 50° C. to obtain each cosmetic sheet. Next, each of the produced cosmetic sheets was affixed onto a blemish (senile lentigo) having an L value of 62, and the blemish concealing properties was visually evaluated.

The evaluation was performed according to the following criteria.
AA: It is possible to conceal the blemish (the color and boundary of the blemish are note visually recognized).
A: The boundary of the blemish is not visually recognized, but the outside of the blemish appears to be slightly different in tint from the center of the blemish when closely and carefully observed.
C: It is not possible to conceal the blemish.

### (Method for Evaluating Fixity (Water Resistance) (Reference Evaluation))

The fixity (water resistance) of the white cosmetic material ink was evaluated by the following method. First, each of the prepared white cosmetic material inks was filled in an ink tank of an inkjet printer equipped with an LB3 inkjet nozzle (manufactured by Panasonic Precision Devices Co., Ltd.). A polylactic acid sheet having a thickness of 400 nm was attached to a support formed of filter paper, and this was prepared as a substrate. Next, the white cosmetic material ink was applied onto the polylactic acid sheet using an inkjet printer to form a circular pattern (fill pattern) having a radius of 30 mm. At this time, the amount of the white cosmetic material ink applied was 10 ul. The sheet after printing was dried in an environment of 50° C. to obtain each printed matter. Next, water was sufficiently poured onto the ink layer of the printed matter so that the printed matter was immersed in the water, and dissolution of the ink was visually observed.

The evaluation was performed according to the following criteria.
AA: The ink does not dissolve in water.
A: Obvious dissolution of the ink is not observed, but a small amount of ink is transferred to a finger when the printed surface is rubbed with the finger.
B: The ink dissolves in the water.

TABLE 3

| | | | Comparative | Comparative | | | Comparative |
|---|---|---|---|---|---|---|---|
| | | | Example | Example | Example | Example | Example |
| | | | 3-1 | 3-2 | 3-1 | 3-2 | 3-3 |
| Ingredients | White | Median diameter (nm) | 175 | 250 | 340 | 881 | 950 |
| of white | pigment (A2) | Content (% by mass) | 10 | 10 | 10 | 10 | 10 |
| cosmetic | | (A2)/{(A2) + (B) + (C) + (D)} × 100 | | | | | |
| material | Acrylic | Content (% by mass) | 7 | 7 | 7 | 7 | 7 |
| ink | particles (B) | (B)/{(A2) + (B) + (C) + (D)} × 100 | | | | | |
| | Alcohol-based | Content (% by mass) | 20 | 20 | 20 | 20 | 20 |
| | solvent (C) | {(C1) + (C2)}/{(A2) + (B) + (C) + (D)} × 100 | | | | | |
| | Dihydric | Content (% by mass) | 10 | 10 | 10 | 10 | 10 |
| | alcohol-based | (C1)/{(A2) + (B) + (C) + (D)} × 100 | | | | | |
| | solvent (C1) | | | | | | |
| | Trihydric | Content (% by mass) | 10 | 10 | 10 | 10 | 10 |
| | alcohol-based | (C2)/{(A2) + (B) + (C) + (D)} × 100 | | | | | |
| | solvent (C2) | | | | | | |
| | Water (D) | Content (% by mass) | 63 | 63 | 63 | 63 | 63 |
| | | (D)/{(A2) + (B) + (C) + (D)} × 100 | | | | | |
| Physical | Viscosity (mPa · s) | | 7.5 | 7.5 | 7.5 | 7.5 | 7.5 |
| properties | | | | | | | |
| Evaluation | Ejection stability | | AA | AA | AA | AA | C |
| | Concealing properties | | C | C | AA | AA | AA |
| Reference | Fixity (water resistance) | | AA | AA | AA | AA | AA |
| evaluation | | | | | | | |

### (Results)

As presented in Table 3 above, when the median diameter of the white pigment (A2) contained in the white cosmetic material ink was 300 nm or more and 900 nm or less, it was possible to favorably conceal discolored areas with the cosmetic sheets produced using the white cosmetic material inks, and the ejection stability in inkjet printing was also favorable (see Examples 3-1 and 3-2).

On the other hand, when the median diameter of the white pigment (A2) contained in the white cosmetic material ink was decreased to 250 nm, it was not possible to favorably conceal discolored areas with the cosmetic sheets produced using the white cosmetic material inks (see Comparative Examples 3-1 and 3-2). When the median diameter of the white pigment (A2) contained in the white cosmetic material ink was increased to 950 nm, the ejection stability in inkjet printing was deteriorated (see Comparative Example 3-3).

In all of the white cosmetic material inks of Examples 3-1 and 3-2 and Comparative Examples 3-1 to 3-3 as well, the fixity (water resistance) was favorable.

### Example 4

In Example 4, each white cosmetic material ink was prepared by varying the content of the white pigment (A2) contained in the white cosmetic material ink. Specifically, the respective white cosmetic material inks were prepared by mixing the respective materials mentioned above at the ingredient ratios in Examples 4-1 to 4-4 and Comparative Examples 4-1 and 4-2 presented in Table 4 below.

### [Evaluation]

The viscosity of each of the prepared white cosmetic material inks was measured by the same method as in Example 3. Furthermore, the ejection stability in inkjet printing, concealing properties for discolored areas, and the fixity (water resistance) of each white cosmetic material ink were also evaluated by the same methods as in Example 3.

TABLE 4

| | | | Compar- | | | | | Compar- |
|---|---|---|---|---|---|---|---|---|
| | | | ative | | | | | ative |
| | | | Example | Example | Example | Example | Example | Example |
| | | | 4-1 | 4-1 | 4-2 | 4-3 | 4-4 | 4-2 |
| Ingredients | White | Median diameter (nm) | 340 | 340 | 340 | 340 | 340 | 340 |
| of white | pigment (A2) | Content (% by mass) | 3 | 5 | 10 | 20 | 30 | 40 |
| cosmetic | | (A2)/{(A2) + (B) + (C) + (D)} × 100 | | | | | | |
| material | Acrylic | Content (% by mass) | 7 | 7 | 7 | 7 | 7 | 7 |
| ink | particles (B) | (B)/{(A2) + (B) + (C) + (D)} × 100 | | | | | | |
| | Alcohol-based | Content (% by mass) | 20 | 20 | 20 | 20 | 20 | 20 |
| | solvent (C) | {(C1) + (C2)}/{(A2) + (B) + (C) + (D)} × 100 | | | | | | |
| | Dihydric | Content (% by mass) | 10 | 10 | 10 | 10 | 10 | 10 |
| | alcohol-based | (C1)/{(A2) + (B) + (C) + (D)} × 100 | | | | | | |
| | solvent (C1) | | | | | | | |
| | Trihydric | Content (% by mass) | 10 | 10 | 10 | 10 | 10 | 10 |
| | alcohol-based | (C2)/{(A2) + (B) + (C) + (D)} × 100 | | | | | | |
| | solvent (C2) | | | | | | | |
| | Water (D) | Content (% by mass) | 70 | 68 | 63 | 53 | 43 | 33 |
| | | (D)/{(A2) + (B) + (C) + (D)} × 100 | | | | | | |
| Physical | Viscosity (mPa · s) | | 2.8 | 3.7 | 7.5 | 11 | 16 | 21 |
| properties | | | | | | | | |
| Evaluation | Ejection stability | | AA | AA | AA | AA | A | C |
| | Concealing properties | | C | A | AA | AA | AA | AA |
| Reference | Fixity (water resistance) | | AA | AA | AA | AA | AA | AA |
| evaluation | | | | | | | | |

### (Results)

As presented in Table 4 above, it was possible to improve the concealing properties of the cosmetic sheets for discolored areas and also to improve the ejection stability in inkjet printing when the content of the white pigment (A2) in the white cosmetic material ink was set to 4% by mass or more and 35% by mass or less with respect to the total amount of the white pigment (A2), acrylic particles (B), alcohol-based solvent (C), and water (D) (see Examples 4-1 to 4-4).

On the other hand, it was not possible to favorably conceal discolored areas with the cosmetic sheet produced using the white cosmetic material ink when the content of the white pigment (A2) contained in the white cosmetic material ink was decreased to 3% by mass (see Comparative Example 4-1). Furthermore, the ejection stability in inkjet printing was deteriorated when the content of the white pigment (A2) in the white cosmetic material ink was increased to 40% by mass (see Comparative Example 4-2).

In all of the white cosmetic material inks of Examples 4-1 to 4-4 and Comparative Examples 4-1 and 4-2 as well, the fixity (water resistance) was favorable.

### Example 5

In Example 5, each white cosmetic material ink was prepared by varying the contents of the dihydric alcohol-based solvent (C1) and trihydric alcohol-based solvent (C2) contained in the white cosmetic material ink. Specifically, the respective white cosmetic material inks were prepared by mixing the respective materials mentioned above at the ingredient ratios in Examples 5-1 to 5-6 and Comparative Examples 5-1 to 5-4 presented in Table 5 below.

### [Evaluation]

The viscosity of each of the prepared white cosmetic material inks was measured by the same method as in Example 3. Furthermore, the ejection stability in inkjet printing, concealing properties for discolored areas, and the fixity (water resistance) of each white cosmetic material ink were also evaluated by the same methods as in Example 3.

TABLE 5

| | | | Comparative | | | | Comparative |
|---|---|---|---|---|---|---|---|
| | | | Example | Example | Example | Example | Example |
| | | | 5-1 | 5-1 | 5-2 | 5-3 | 5-2 |
| Ingredients | White | Median diameter (nm) | 340 | 340 | 340 | 340 | 340 |
| of white | pigment (A2) | Content (% by mass) | 10 | 10 | 10 | 10 | 10 |
| cosmetic | | (A2)/{(A2) + (B) + (C) + (D)} × 100 | | | | | |
| material | Acrylic | Content (% by mass) | 7 | 7 | 7 | 7 | 7 |
| ink | particles (B) | (B)/{(A2) + (B) + (C) + (D)} × 100 | | | | | |
| | Alcohol-based | Content (% by mass) | 15 | 20 | 30 | 50 | 60 |
| | solvent (C) | {(C1) + (C2)}/{(A2) + (B) + (C) + (D)} × 100 | | | | | |
| | Dihydric | Content (% by mass) | 5 | 10 | 20 | 40 | 50 |
| | alcohol-based | (C1)/{(A2) + (B) + (C) + (D)} × 100 | | | | | |
| | solvent (C1) | | | | | | |
| | Trihydric | Content (% by mass) | 10 | 10 | 10 | 10 | 10 |
| | alcohol-based | (C2)/{(A2) + (B) + (C) + (D)} × 100 | | | | | |
| | solvent (C2) | | | | | | |
| | Water (D) | Content (% by mass) | 68 | 63 | 53 | 33 | 23 |
| | | (D)/{(A2) + (B) + (C) + (D)} × 100 | | | | | |
| Physical | Viscosity (mPa · s) | | 2.9 | 7.5 | 9.8 | 17 | 21 |
| properties | | | | | | | |
| Evaluation | Ejection stability | | C | AA | AA | AA | C |
| | Concealing properties | | AA | AA | AA | AA | AA |
| Reference | Fixity (water resistance) | | AA | AA | AA | AA | AA |
| evaluation | | | | | | | |

| | | | Comparative | | | | Comparative |
|---|---|---|---|---|---|---|---|
| | | | Example | Example | Example | Example | Example |
| | | | 5-3 | 5-4 | 5-5 | 5-6 | 5-4 |
| Ingredients | White | Median diameter (nm) | 340 | 340 | 340 | 340 | 340 |
| of white | pigment (A2) | Content (% by mass) | 10 | 10 | 10 | 10 | 10 |
| cosmetic | | (A2)/{(A2) + (B) + (C) + (D)} × 100 | | | | | |
| material | Acrylic | Content (% by mass) | 7 | 7 | 7 | 7 | 7 |
| ink | particles (B) | (B)/{(A2) + (B) + (C) + (D)} × 100 | | | | | |
| | Alcohol-based | Content (% by mass) | 15 | 20 | 30 | 50 | 60 |
| | solvent (C) | {(C1) + (C2)}/{(A2) + (B) + (C) + (D)} × 100 | | | | | |
| | Dihydric | Content (% by mass) | 10 | 10 | 10 | 10 | 10 |
| | alcohol-based | (C1)/{(A2) + (B) + (C) + (D)} × 100 | | | | | |
| | solvent (C1) | | | | | | |
| | Trihydric | Content (% by mass) | 5 | 10 | 20 | 40 | 50 |
| | alcohol-based | (C2)/{(A2) + (B) + (C) + (D)} × 100 | | | | | |
| | solvent (C2) | | | | | | |
| | Water (D) | Content (% by mass) | 68 | 63 | 53 | 33 | 23 |
| | | (D)/{(A2) + (B) + (C) + (D)} × 100 | | | | | |
| Physical | Viscosity (mPa · s) | | 2.9 | 7.5 | 9.8 | 17 | 21 |
| properties | | | | | | | |
| Evaluation | Ejection stability | | C | AA | AA | AA | C |
| | Concealing properties | | AA | AA | AA | AA | AA |
| Reference | Fixity (water resistance) | | AA | AA | AA | AA | AA |
| evaluation | | | | | | | |

### (Results)

As presented in Table 5 above, it was possible to improve the ejection stability in inkjet printing when the content of the alcohol-based solvent (C) was set to 17% by mass or more and 55% by mass or less with respect to the total amount of the white pigment (A2), acrylic particles (B), alcohol-based solvent (C), and water (D) (see Examples 5-1 to 5-6).

On the other hand, the ejection stability in inkjet printing was deteriorated when the content of the alcohol-based solvent (C) was decreased to 15% by mass (see Comparative Examples 5-1 and 5-3). Furthermore, the ejection stability in inkjet printing was deteriorated when the content of the alcohol-based solvent (C) was increased to 60% by mass as well (see Comparative Examples 5-2 and 5-4).

In all of the white cosmetic material inks of Examples 5-1 to 5-6 and Comparative Examples 5-1 to 5-4 as well, the fixity (water resistance) was favorable.

### Example 6

In Example 6, each white cosmetic material ink was prepared by varying the content of the acrylic particles (B) contained in the white cosmetic material ink although the content of the acrylic particles (B) is not related to the effects, such as concealing properties and ejection stability in inkjet printing, of the white cosmetic material ink according to the present invention. Specifically, the respective white cosmetic material inks were prepared by mixing the respective materials mentioned above at the ingredient ratios in Examples 6-1 to 6-6 presented in Table 6 below.

### [Evaluation]

The viscosity of each of the prepared white cosmetic material inks was measured by the same method as in Example 3. Furthermore, the ejection stability in inkjet printing, concealing properties for discolored areas, and the fixity (water resistance) of each white cosmetic material ink were also evaluated by the same methods as in Example 3.

TABLE 6

| | | | Example | Example | Example | Example | Example | Example |
|---|---|---|---|---|---|---|---|---|
| | | | 6-1 | 6-2 | 6-3 | 6-4 | 6-5 | 6-6 |
| Ingredients | White | Median diameter (nm) | 340 | 340 | 340 | 340 | 340 | 340 |
| of white | pigment (A2) | Content (% by mass) | 10 | 10 | 10 | 10 | 10 | 10 |
| cosmetic | | (A2)/{(A2) + (B) + (C) + (D)} × 100 | | | | | | |
| material | Acrylic | Content (% by mass) | 2.5 | 3 | 5 | 7 | 10 | 20 |
| ink | particles (B) | (B)/{(A2) + (B) + (C) + (D)} × 100 | | | | | | |
| | Alcohol-based | Content (% by mass) | 20 | 20 | 20 | 20 | 20 | 20 |
| | solvent (C) | {(C1) + (C2)}/{(A2) + (B) + (C) + (D)} × 100 | | | | | | |
| | Dihydric | Content (% by mass) | 10 | 10 | 10 | 10 | 10 | 10 |
| | alcohol-based | (C1)/{(A2) + (B) + (C) + (D)} × 100 | | | | | | |
| | solvent (C1) | | | | | | | |
| | Trihydric | Content (% by mass) | 10 | 10 | 10 | 10 | 10 | 10 |
| | alcohol-based | (C2)/{(A2) + (B) + (C) + (D)} × 100 | | | | | | |
| | solvent (C2) | | | | | | | |
| | Water (D) | Content (% by mass) | 67.5 | 67 | 65 | 63 | 60 | 50 |
| | | (D)/{(A2) + (B) + (C) + (D)} × 100 | | | | | | |
| Physical | Viscosity (mPa · s) | | 3.7 | 4.2 | 4.6 | 7.5 | 10 | 19 |
| properties | | | | | | | | |
| Evaluation | Ejection stability | | AA | AA | AA | AA | AA | A |
| | Concealing properties | | AA | AA | AA | AA | AA | AA |
| Reference | Fixity (water resistance) | | B | A | AA | AA | AA | AA |
| evaluation | | | | | | | | |

### (Results)

As presented in Table 6 above, it was possible to reliably improve not only the ejection stability in inkjet printing and concealing properties for discolored areas of the white cosmetic material inks but also the fixity (water resistance) when the content of the acrylic particles (B) was set to 3% by mass or more with respect to the total amount of the white pigment (A2), acrylic particles (B), alcohol-based solvent (C), and water (D).

This application is based on Japanese Patent Application Nos. 2022-071815 and 2022-071816, filed on Apr. 25, 2022, the contents of which are incorporated herein.

The embodiments and Examples disclosed herein should be considered as illustrative and not restrictive in all respects. The scope of the present invention is defined by the claims, rather than the above description, and is intended to include all modifications within the meaning and scope of the claims and their equivalent concepts.

The cosmetic sheet to which the cosmetic material ink in the present invention is applied can be used for various purposes, for example, medical purposes to make discolored areas less noticeable and cosmetic purposes such as skin coloring, beautification, and makeup.

1. A cosmetic material ink that is a blue cosmetic material ink comprising:
a blue pigment (A1);
acrylic particles (B);
an alcohol-based solvent (C); and
water (D),
wherein a content of the blue pigment (A1) is 0.5% by mass or more and 4.5% by mass or less with respect to a total amount of the blue pigment (A1), the acrylic particles (B), the alcohol-based solvent (C), and the water (D),
the alcohol-based solvent (C) includes a dihydric alcohol-based solvent (C1) and a trihydric alcohol-based solvent (C2), and
a total content of the dihydric alcohol-based solvent (C1) and the trihydric alcohol-based solvent (C2) is 30% by mass or more and 85% by mass or less with respect to the total amount of the blue pigment (A1), the acrylic particles (B), the alcohol-based solvent (C), and the water (D).

2. The cosmetic material ink according to claim 1, wherein a content of the dihydric alcohol-based solvent (C1) is 15% by mass or more and 45% by mass or less and a content of the trihydric alcohol-based solvent (C2) is 15% by mass or more and 45% by mass or less with respect to the total amount of the blue pigment (A1), the acrylic particles (B), the alcohol-based solvent (C), and the water (D).

3. The cosmetic material ink according to claim 1, wherein the blue pigment (A1) includes at least one selected from Prussian blue (ferric ferrocyanide), ultramarine blue, lapis lazuli, blue verditer, aluminum-cobalt oxide, aluminum-zinc-cobalt oxide, silicon-cobalt oxide, silicon-zinc-cobalt oxide, cobalt pigment, smalt, cobalt blue, cobalt stannate, cobalt chrome blue, cobalt-aluminum-silicon oxide, and manganese blue.

4. The cosmetic material ink according to claim 1, wherein the blue pigment (A1) has a median diameter of 90 nm or more and 500 nm or less.

5. The cosmetic material ink according to claim 1, which has a viscosity of 3 mPa·s or more and 15 mPa·s or less at 25° C. when the viscosity is measured at a shear rate of 1000 (1/s) using a cone-plate viscometer.

6. The cosmetic material ink according to claim 1, wherein the alcohol-based solvent (C) includes an alcohol-based solvent having a boiling point of 200° C. or more.

7. The cosmetic material ink according to claim 1, wherein the dihydric alcohol-based solvent (C1) contains at least one selected from diethylene glycol, propylene glycol, 1,3-propanediol, butylene glycol, and hexanediol.

8. The cosmetic material ink according to claim 1, wherein the acrylic particles (B) contain at least one of a homopolymer of a (meth)acrylic monomer and a copolymer of two or more kinds of (meth)acrylic monomers.

9. The cosmetic material ink according to claim 1, which is an ink for inkjet printing to be applied to a sheet having a thickness of 10 nm or more and 3000 nm or less by inkjet printing.

10. A cosmetic sheet comprising:
a sheet having a thickness of 10 nm or more and 3000 nm or less; and
a coating film that is formed on the sheet and has at least an ink layer formed of the cosmetic material ink according to claim 1.

11. A cosmetic material ink that is a white cosmetic material ink comprising:
a white pigment (A2);
acrylic particles (B);
an alcohol-based solvent (C); and
water (D),
wherein the white pigment (A2) has a median diameter of 300 nm or more and 900 nm or less,
a content of the white pigment (A2) is 4% by mass or more and 35% by mass or less with respect to a total amount of the white pigment (A2), the acrylic particles (B), the alcohol-based solvent (C), and the water (D),
the alcohol-based solvent (C) includes a dihydric alcohol-based solvent (C1) and a trihydric alcohol-based solvent (C2), and
a total content of the dihydric alcohol-based solvent (C1) and the trihydric alcohol-based solvent (C2) is 17% by mass or more and 55% by mass or less with respect to the total amount of the white pigment (A2), the acrylic particles (B), the alcohol-based solvent (C), and the water (D).

12. The cosmetic material ink according to claim 11, wherein a content of the acrylic particles (B) is 3% by mass or more and 20% by mass or less with respect to the total amount of the white pigment (A2), the acrylic particles (B), the alcohol-based solvent (C), and the water (D).

13. The cosmetic material ink according to claim 11, which has a viscosity of 3 mPa·s or more and 20 mPa·s or less at 25° C. when the viscosity is measured at a shear rate of 1000 (1/s) using a cone-plate viscometer.

14. The cosmetic material ink according to claim 11, wherein a content of the dihydric alcohol-based solvent (C1) is 10% by mass or more and 40% by mass or less and a content of the trihydric alcohol-based solvent (C2) is 7% by mass or more and 45% by mass or less with respect to the total amount of the white pigment (A2), the acrylic particles (B), the alcohol-based solvent (C), and the water (D).

15. The cosmetic material ink according to claim 11, wherein the white pigment (A2) includes at least one selected from titanium oxide, zinc oxide, cerium oxide, barium sulfate, talc, muscovite, phlogopite, red mica, biotite, synthetic mica, sericite, synthetic sericite, kaolin, silicon carbide, bentonite, smectite, silicic anhydride, aluminum oxide, magnesium oxide, zirconium oxide, antimony oxide, diatomaceous earth, aluminum silicate, magnesium aluminum metasilicate, calcium silicate, barium silicate, magnesium silicate, calcium carbonate, magnesium carbonate, hydroxyapatite, and boron nitride.

16. The cosmetic material ink according to claim 11, wherein the alcohol-based solvent (C) includes an alcohol-based solvent having a boiling point of 200° C. or more.

17. The cosmetic material ink according to claim 11, wherein the dihydric alcohol-based solvent (C1) contains at least one selected from diethylene glycol, propylene glycol, 1,3-propanediol, butylene glycol, and hexanediol.

18. The cosmetic material ink according to claim 11, wherein the acrylic particles (B) contain at least one of a homopolymer of a (meth)acrylic monomer and a copolymer of two or more kinds of (meth)acrylic monomers.

19. The cosmetic material ink according to claim 11, which is an ink for inkjet printing to be applied to a sheet having a thickness of 10 nm or more and 3000 nm or less by inkjet printing.

20. A cosmetic sheet comprising:
a sheet having a thickness of 10 nm or more and 3000 nm or less; and
a coating film that is formed on the sheet and has at least an ink layer formed of the cosmetic material ink according to claim 11.
